(19) United States
(12) Patent Application Publication
(10) Pub. No.: US 2025/0066438 A1
(43) Pub. Date: Feb. 27, 2025

(54) METHODS AND COMPOSITIONS FOR PREVENTING OR TREATING CANCER

(71) Applicant: Colossal Biosciences Inc., Austin, TX (US)

(72) Inventors: Eriona HYSOLLI, Austin, TX (US); Evan APPLETON, Austin, TX (US); Ben E. LAMM, Austin, TX (US)

(21) Appl. No.: 18/813,984

(22) Filed: Aug. 23, 2024

Related U.S. Application Data
(60) Provisional application No. 63/578,273, filed on Aug. 23, 2023.

(51) Int. Cl. C07K 14/47; A61K 38/00; A61P 35/00; C07K 14/54

(52) U.S. Cl. CPC C07K 14/4746; A61P 35/00; C07K 14/5415; A61K 38/00

(57) ABSTRACT

Provided herein are methods of treating or preventing cancer in a subject in need thereof, the methods comprise administering to the subject in need thereof (a) one or more nucleic acids encoding an elephant p53 protein, an elephant LIF protein, and/or combinations thereof, or (b) one or more elephant p53 proteins, elephant LIF proteins, and/or combinations thereof. Also provided are pharmaceutical compositions comprising a pharmaceutically acceptable carrier and (a) one or more nucleic acids encoding an elephant p53 protein, an elephant LIF protein, and/or combinations thereof, or (b) one or more elephant p53 proteins, elephant LIF proteins, and/or combinations thereof.

### CROSS REFERENCE TO RELATED APPLICATION

The application claims priority to U.S. Provisional Application No. 63/578,273, filed Aug. 23, 2023, the disclosure of which is incorporated herein by reference in its entirety.

### FIELD OF THE INVENTION

The present disclosure generally relates to methods of treating or preventing cancer in a subject in need thereof. The methods comprise administering to the subject in need thereof (a) one or more nucleic acids encoding an elephant p53 protein, an elephant LIF protein, and/or combinations thereof; or (b) one or more elephant p53 proteins, elephant LIF proteins, and/or combinations thereof. Also provided are pharmaceutical compositions comprising a pharmaceutically acceptable carrier and (a) one or more nucleic acids encoding an elephant p53 protein, an elephant LIF protein, and/or combinations thereof, or (b) one or more elephant p53 proteins, elephant LIF proteins, and/or combinations thereof. Also provided are kits for treating or preventing cancer in a subject in need thereof.

### REFERENCE TO SEQUENCE LISTING SUBMITTING ELECTRONICALLY

This application contains a sequence listing, which is submitted electronically. The contents of the electronic sequence listing (069296.14US2 Sequence Lisitng.xml; size: 179,784 bytes; and creation date of Aug. 22, 2024) is herein incorporated by reference in its entirety.

### BACKGROUND OF THE INVENTION

The tumor suppressor protein p53 (TP53) gene encodes for the p53 protein, one or the main regulators of cell division and cell death. It is activated and stabilized after a variety of stresses, which can include radiation, toxic substances, hypoxia, high production of Reactive Oxygen Species (ROS), uncontrolled cell cycle, and others (Capuozzo et al., Biology 11:1325 (2022)). P53 can trigger cell cycle arrest, DNA repair, senescence, apoptosis, and autophagy. It also has documented roles in pluripotency and the germ line (Fu et al., Protein Cell 11(1):71-78 (2020)). Mutations in TP53 are found in approximately 50% of tumors, and these mutations are under positive selection since they are highly beneficial for cell survival and proliferation (Kennedy and Lowe, Cell Death Differ. 29:983-87 (2022); Levine, Nat. Rev. Cancer 20:471-80 (2020), Levine, Oncogene 40:5975-5983 (2021)). In addition to p53, irregularities in other tumor suppressor genes, such as interleukin-6 class cytokine leukemia inhibitory factor (LIF) have been associated with oncogenesis (Caulin, et al. Trends Ecol. Evol. (2011); Vazquez et al., Cell Rep. (2018)).

Humans have one copy of TP53; however, elephants comprise at least 20 polymorphic copies of TP53 and 8 polymorphic copies of LIF, which are both believed to be a major reason that there are very low cancer rates in elephants.

Thus, there remains a need for compositions and methods to deliver elephant p53 and/or elephant LIF function to cells to treat or prevent cancers.

### BRIEF SUMMARY OF THE INVENTION

Provided herein are methods of treating or preventing cancer in a subject in need thereof. The methods comprising administering to the subject in need thereof (a) one or more nucleic acids encoding an elephant p53 protein, wherein the one or more nucleic acids encoding the elephant p53 protein comprise a nucleotide sequence having at least 80%, at least 85%, at least 90%, at least 91%, at least 92%, at least 93%, at least 94%, at least 95%, at least 96%, at least 97%, at least 98%, or at least 99% identity with a nucleotide sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, and combinations thereof; or (b) one or more elephant p53 proteins, wherein the one or more elephant p53 proteins comprise an amino acid sequence with at least 85%, at least 90%, at least 91%, at least 92%, at least 93%, at least 94%, at least 95%, at least 96%, at least 97%, at least 98%, or at least 99% identity to an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NOs:75-133, and combinations thereof.

In certain embodiments, the one or more nucleic acids encoding the elephant p53 protein comprise a nucleotide sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, and combinations thereof. In certain embodiments, the one or more elephant p53 proteins comprise an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NOs:75-133, and combinations thereof.

In certain embodiments, the subject in need thereof is administered (a) one, two, three, four, five, six, seven, eight, nine, or ten nucleic acids encoding an elephant p53 protein; or (b) one, two, three, four, five six, seven, eight, nine, or ten elephant p53 proteins.

In certain embodiments, the subject in need thereof is further administered (c) one or more nucleic acids encoding an elephant leukemia inhibitory factor (LIF) protein, wherein the one or more nucleic acids encoding the elephant LIF protein comprise a nucleotide sequence having at least 80%, at least 85%, at least 90%, at least 91%, at least 92%, at least 93%, at least 94%, at least 95%, at least 96%, at least 97%, at least 98%, or at least 99% identity with a nucleotide sequence selected from the group consisting of SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, and combinations thereof; or (d) one or more elephant LIF proteins, wherein the one or more elephant LIF proteins comprise an amino acid sequence with at least 85%, at least 90%, at least 91%, at least 92%, at least 93%, at least 94%, at least 95%, at least 96%, at least 97%, at least 98%, or at least 99% identity to an amino acid sequence selected from the group consisting of SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NOs:134-140, and combinations thereof

In certain embodiments, the one or more nucleic acids encoding the elephant LIF protein comprise a nucleotide sequence selected from the group consisting of SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, and combinations thereof. In certain embodiments, the one or more elephant LIF proteins comprise an amino acid sequence selected from the group consisting of SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NOs:134-140, and combinations thereof.

In certain embodiments, the subject in need thereof is administered (c) one, two, three, four, five, six, seven, eight, nine, or ten nucleic acids encoding an elephant LIF protein; or (d) one, two, three, four, five, six, seven, eight, nine, or ten elephant LIF proteins.

Also provided are methods of treating or preventing cancer in a subject in need thereof, the methods comprising administering to the subject in need thereof (a) one or more nucleic acids encoding an elephant leukemia inhibitory factor (LIF) protein, wherein the one or more nucleic acids encoding the elephant LIF protein comprise a nucleotide sequence having at least 80% identity with a nucleotide sequence selected from the group consisting of SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, and combinations thereof; or (b) one or more elephant LIF proteins, wherein the one or more elephant LIF proteins comprise an amino acid sequence with at least 85% identity to an amino acid sequence selected from the group consisting of SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NOs:134-140, and combinations thereof.

In certain embodiments, the one or more nucleic acids encoding the elephant LIF protein comprise a nucleotide sequence selected from the group consisting of SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, and combinations thereof. In certain embodiments, the one or more elephant LIF proteins comprise an amino acid sequence selected from the group consisting of SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NOs:134-140, and combinations thereof.

In certain embodiments, the subject in need thereof is administered (a) one, two, three, four, five, six, seven, eight, nine, or ten nucleic acids encoding an elephant LIF protein; or (b) one, two, three, four, five, six, seven, eight, nine, or ten elephant LIF proteins.

In certain embodiments, the one or more nucleic acids or the one or more proteins are in the form of a composition, wherein the composition further comprises a pharmaceutically acceptable carrier.

In certain embodiments, the subject in need thereof is a mammal. The mammal can, for example, be a human.

In certain embodiments, the cancer is selected from a lung cancer, a gastric cancer, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, and other solid tumors, and a non-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

Also provided are pharmaceutical compositions comprising a pharmaceutically acceptable carrier and (a) one or more nucleic acids encoding an elephant p53 protein, wherein the one or more nucleic acids encoding the elephant p53 protein comprise a nucleotide sequence having at least 80%, at least 85%, at least 90%, at least 91%, at least 92%, at least 93%, at least 94%, at least 95%, at least 96%, at least 97%, at least 98%, or at least 99% identity with a nucleotide sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, and combinations thereof; or (b) one or more elephant p53 proteins, wherein the one or more elephant p53 proteins comprise an amino acid sequence with at least 85%, at least 90%, at least 91%, at least 92%, at least 93%, at least 94%, at least 95%, at least 96%, at least 97%, at least 98%, or at least 99% identity to an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NOs:75-133, and combinations thereof.

In certain embodiments, the one or more nucleic acids encoding the elephant p53 protein comprise a nucleotide sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, and combinations thereof. In certain embodiments, the one or more elephant p53 proteins comprise an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NOs:75-133, and combinations thereof.

In certain embodiments, the pharmaceutical composition comprises (a) one, two, three, four, five, six, seven, eight, nine, or ten nucleic acids encoding an elephant p53 protein; or (b) one, two, three, four, five six, seven, eight, nine, or ten elephant p53 proteins.

In certain embodiments, the pharmaceutical composition further comprises (c) one or more nucleic acids encoding an elephant leukemia inhibitory factor (LIF) protein, wherein the one or more nucleic acids encoding the elephant LIF protein comprise a nucleotide sequence having at least 80%, at least 85%, at least 90%, at least 91%, at least 92%, at least 93%, at least 94%, at least 95%, at least 96%, at least 97%, at least 98%, or at least 99% identity with a nucleotide sequence selected from the group consisting of SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, and combinations thereof; or (d) one or more elephant LIF proteins, wherein the one or more elephant LIF proteins comprise an amino acid sequence with at least 85%, at least 90%, at least 91%, at least 92%, at least 93%, at least 94%, at least 95%, at least 96%, at least 97%, at least 98%, or at least 99% identity to an amino acid sequence selected from the group consisting of SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NOs:134-140, and combinations thereof

In certain embodiments, the one or more nucleic acids encoding the elephant LIF protein comprise a nucleotide sequence selected from the group consisting of SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, and combinations thereof. In certain embodiments, the one or more elephant LIF proteins comprise an amino acid sequence selected from the group consisting of SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NOs:134-140, and combinations thereof.

In certain embodiments, the pharmaceutical composition comprises (c) one, two, three, four, five, six, seven, eight, nine, or ten nucleic acids encoding an elephant LIF protein; or (d) one, two, three, four, five, six, seven, eight, nine, or ten elephant LIF proteins.

Also provided are pharmaceutical compositions comprising a pharmaceutically acceptable carrier and (a) one or more nucleic acids encoding an elephant leukemia inhibitory factor (LIF) protein, wherein the one or more nucleic acids encoding the elephant LIF protein comprise a nucleotide sequence having at least 80% identity with a nucleotide sequence selected from the group consisting of SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, and combinations thereof; or (b) one or more elephant LIF proteins, wherein the one or more elephant LIF proteins comprise an amino acid sequence with at least 85% identity to an amino acid sequence selected from the group consisting of SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NOs:134-140, and combinations thereof.

In certain embodiments, the one or more nucleic acids encoding the elephant LIF protein comprise a nucleotide sequence selected from the group consisting of SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, and combinations thereof. In certain embodiments, the one or more elephant LIF proteins comprise an amino acid sequence selected from the group consisting of SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NOs:134-140, and combinations thereof.

In certain embodiments, the pharmaceutical composition comprises (a) one, two, three, four, five, six, seven, eight, nine, or ten nucleic acids encoding an elephant LIF protein; or (b) one, two, three, four, five, six, seven, eight, nine, or ten elephant LIF proteins.

Also provided are kits for treating or preventing cancer in a subject in need thereof. The kits comprise (a) one or more nucleic acids encoding an elephant p53 protein, wherein the one or more nucleic acids encoding the elephant p53 protein comprise a nucleotide sequence having at least 80%, at least 85%, at least 90%, at least 91%, at least 92%, at least 93%, at least 94%, at least 95%, at least 96%, at least 97%, at least 98%, or at least 99% identity with a nucleotide sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, and combinations thereof; or (b) one or more elephant p53 proteins, wherein the one or more elephant p53 proteins comprise an amino acid sequence with at least 85%, at least 90%, at least 91%, at least 92%, at least 93%, at least 94%, at least 95%, at least 96%, at least 97%, at least 98%, or at least 99% identity to an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NOs:75-133, and combinations thereof, and (c) instructions for use.

In certain embodiments, the one or more nucleic acids encoding the elephant p53 protein comprise a nucleotide sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, and combinations thereof. In certain embodiments, the one or more elephant p53 proteins comprise an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NOs:75-133, and combinations thereof.

In certain embodiments, the kit comprises (a) one, two, three, four, five, six, seven, eight, nine, or ten nucleic acids encoding an elephant p53 protein; or (b) one, two, three, four, five six, seven, eight, nine, or ten elephant p53 proteins.

In certain embodiments, the kit further comprises (c) one or more nucleic acids encoding an elephant leukemia inhibitory factor (LIF) protein, wherein the one or more nucleic acids encoding the elephant LIF protein comprise a nucleotide sequence having at least 80%, at least 85%, at least 90%, at least 91%, at least 92%, at least 93%, at least 94%, at least 95%, at least 96%, at least 97%, at least 98%, or at least 99% identity with a nucleotide sequence selected from the group consisting of SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, and combinations thereof; or (d) one or more elephant LIF proteins, wherein the one or more elephant LIF proteins comprise an amino acid sequence with at least 85%, at least 90%, at least 91%, at least 92%, at least 93%, at least 94%, at least 95%, at least 96%, at least 97%, at least 98%, or at least 99% identity to an amino acid sequence selected from the group consisting of SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NOs:134-140, and combinations thereof.

In certain embodiments, the one or more nucleic acids encoding the elephant LIF protein comprise a nucleotide sequence selected from the group consisting of SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, and combinations thereof. In certain embodiments, the one or more elephant LIF proteins comprise an amino acid sequence selected from the group consisting of SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NOs:134-140, and combinations thereof.

In certain embodiments, the kit comprises (c) one, two, three, four, five, six, seven, eight, nine, or ten nucleic acids encoding an elephant LIF protein; or (d) one, two, three, four, five, six, seven, eight, nine, or ten elephant LIF proteins.

Also provided are kits for treating or preventing cancer in a subject in need thereof. The kits comprise (a) one or more nucleic acids encoding an elephant leukemia inhibitory factor (LIF) protein, wherein the one or more nucleic acids encoding the elephant LIF protein comprise a nucleotide sequence having at least 80%, at least 85%, at least 90%, at least 91%, at least 92%, at least 93%, at least 94%, at least 95%, at least 96%, at least 97%, at least 98%, or at least 99% identity with a nucleotide sequence selected from the group consisting of SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, and combinations thereof; or (b) one or more elephant LIF proteins, wherein the one or more elephant LIF proteins comprise an amino acid sequence with at least 85%, at least 90%, at least 91%, at least 92%, at least 93%, at least 94%, at least 95%, at least 96%, at least 97%, at least 98%, or at least 99% identity to an amino acid sequence selected from the group consisting of SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NOs:134-140, and combinations thereof.

In certain embodiments, the one or more nucleic acids encoding the elephant LIF protein comprise a nucleotide sequence selected from the group consisting of SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, and combinations thereof. In certain embodiments, the one or more elephant LIF proteins comprise an amino acid sequence selected from the group consisting of SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NOs:134-140, and combinations thereof.

In certain embodiments, the kit comprises (a) one, two, three, four, five, six, seven, eight, nine, or ten nucleic acids encoding an elephant LIF protein; or (b) one, two, three, four, five, six, seven, eight, nine, or ten elephant LIF proteins.

In certain embodiments, the one or more nucleic acids or the one or more proteins are in the form of a composition, and wherein the composition further comprises a pharmaceutically acceptable carrier.

In certain embodiments, the subject in need thereof is a mammal. The mammal can, for example, be a human.

In certain embodiments, the cancer is selected from a lung cancer, a gastric cancer, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, and other solid tumors, and a non-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

### BRIEF DESCRIPTION OF THE DRAWINGS

The foregoing summary, as well as the following detailed description of preferred embodiments of the present application, will be better understood when read in conjunction with the appended drawing. It should be understood, however, that the application is not limited to the precise embodiments shown in the drawings.

FIGS. 1A-1C show alignments of elephant p53 and LIF retrogene open reading frames. FIG. 1A shows an alignment of p53 retrogene 1, 12, 14, 2.2, 2.3, 2.4, 2, 6.2, 6.3, 6.4, 6.5, 6.6, 6.7, 6.8, 6, 8.2, 8.3, 8.4, and 8.5. FIG. 1B shows an alignment of 10.2, 10.3, 10.5, 10.7, and 10. FIG. 1C shows an alignment of the LIF open reading frames.

FIG. 2 shows a graph demonstrating the percent of cells positive for annexin V, propidium iodide (PI), or combination of annexin V and PI in cells that were transfected with TP53 or LIF retrogene expression vectors.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2111.03.

As used herein, “subject” means any animal, preferably a mammal, most preferably a human. The term “mammal” as used herein, encompasses any mammal. Examples of mammals include, but are not limited to, cows, horses, sheep, pigs, cats, dogs, mice, rats, rabbits, guinea pigs, monkeys, humans, etc., more preferably a human.

The words “right,” “left,” “lower,” and “upper” designate directions in the drawings to which reference is made.

It should also be understood that the terms “about,” “approximately,” “generally,” “substantially” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The terms “identical” or percent “identity,” in the context of two or more nucleic acids or polypeptide sequences refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215:403-410 and Altschul et al. (1997) Nucleic Acids Res. 25:3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

As used herein, the term “vector” is a replicon in which another nucleic acid segment can be operably inserted so as to bring about the replication or expression of the segment.

The term “expression” as used herein, refers to the biosynthesis of a gene product. The term encompasses the transcription of a gene into RNA. The term also encompasses translation of RNA into one or more polypeptides, and further encompasses all naturally occurring post-transcriptional and post-translational modifications.

As used herein, the terms “peptide,” “polypeptide,” or “protein” can refer to a molecule comprised of amino acids and can be recognized as a protein by those of skill in the art. The conventional one-letter or three-letter code for amino acid residues is used herein. The terms “peptide,” “polypeptide,” and “protein” can be used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

The peptide sequences described herein are written according to the usual convention whereby the N-terminal region of the peptide is on the left and the C-terminal region is on the right. Although isomeric forms of the amino acids are known, it is the L-form of the amino acid that is represented unless otherwise expressly indicated.

As used herein, the term “isolated” means a biological component (such as a nucleic acid, peptide or protein) has been substantially separated, produced apart from, or purified away from other biological components of the organism in which the component naturally occurs, i.e., other chromosomal and extrachromosomal DNA and RNA, and proteins. Nucleic acids, peptides and proteins that have been “isolated” thus include nucleic acids and proteins purified by standard purification methods. “Isolated” nucleic acids, peptides and proteins can be part of a composition and still be isolated if the composition is not part of the native environment of the nucleic acid, peptide, or protein. The term also embraces nucleic acids, peptides and proteins prepared by recombinant expression in a host cell as well as chemically synthesized nucleic acids.

As used herein, “gene” refers to a nucleic acid comprising an open reading frame encoding a polypeptide, including both exon and (optionally) intron sequences.

As used herein, the elephant p53 and elephant LIF sequences are derived from Elephas maximus.

### Methods of Use

In a general aspect, the invention relates to a method of treating or preventing cancer in a subject in need thereof. The methods comprise administering to the subject in need thereof (a) one or more nucleic acids encoding an elephant p53 protein, wherein the one or more nucleic acids encoding the elephant p53 protein comprise a nucleotide sequence having at least 80%, at least 85%, at least 90%, at least 91%, at least 92%, at least 93%, at least 94%, at least 95%, at least 96%, at least 97%, at least 98%, or at least 99% identity with a nucleotide sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, and combinations thereof; or (b) one or more elephant p53 proteins, wherein the one or more elephant p53 proteins comprise an amino acid sequence with at least 85%, at least 90%, at least 91%, at least 92%, at least 93%, at least 94%, at least 95%, at least 96%, at least 97%, at least 98%, or at least 99% identity to an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NOs:75-133, and combinations thereof.

In certain embodiments, the one or more nucleic acids encoding the elephant p53 protein comprise a nucleotide sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, and combinations thereof. In certain embodiments, the one or more elephant p53 proteins comprise an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ SEQ ID NO:50, SEQ ID NOs:75-133, and combinations thereof.

In certain embodiments, the subject in need thereof is administered (a) one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, or twenty-five nucleic acids encoding an elephant p53 protein; or (b) one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, thirty-six, thirty-seven, thirty-eight, thirty-nine, forty, forty-one, forty-two, forty-three, forty-four, forty-five, forty-six, forty-seven, forty-eight, forty-nine, or fifty elephant p53 proteins.

In certain embodiments, the subject in need thereof is further administered (c) one or more nucleic acids encoding an elephant leukemia inhibitory factor (LIF) protein, wherein the one or more nucleic acids encoding the elephant LIF protein comprise a nucleotide sequence having at least 80%, at least 85%, at least 90%, at least 91%, at least 92%, at least 93%, at least 94%, at least 95%, at least 96%, at least 97%, at least 98%, or at least 99% identity with a nucleotide sequence selected from the group consisting of SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, and combinations thereof; or (d) one or more elephant LIF proteins, wherein the one or more elephant LIF proteins comprise an amino acid sequence with at least 85%, at least 90%, at least 91%, at least 92%, at least 93%, at least 94%, at least 95%, at least 96%, at least 97%, at least 98%, or at least 99% identity to an amino acid sequence selected from the group consisting of SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NOs:134-140, and combinations thereof

In certain embodiments, the one or more nucleic acids encoding the elephant LIF protein comprise a nucleotide sequence selected from the group consisting of SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, and combinations thereof. In certain embodiments, the one or more elephant LIF proteins comprise an amino acid sequence selected from the group consisting of SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NOs:134-140, and combinations thereof.

In certain embodiments, the subject in need thereof is administered (c) one, two, three, four, five, six, seven, eight, nine, ten, eleven, or twelve nucleic acids encoding an elephant LIF protein; or (d) one, two, three, four, five, six, seven, eight, nine, ten, eleven, twelve, thirteen, fourteen, fifteen, sixteen, seventeen, eighteen, or nineteen elephant LIF proteins.

In another general aspect, the invention relates to a method of treating or preventing cancer in a subject in need thereof. The methods can, for example, comprise administering to the subject in need thereof (a) one or more nucleic acids encoding an elephant leukemia inhibitory factor (LIF) protein, wherein the one or more nucleic acids encoding the elephant LIF protein comprise a nucleotide sequence having at least 80% identity with a nucleotide sequence selected from the group consisting of SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, and combinations thereof; or (b) one or more elephant LIF proteins, wherein the one or more elephant LIF proteins comprise an amino acid sequence with at least 85% identity to an amino acid sequence selected from the group consisting of SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NOs:134-140, and combinations thereof.

In certain embodiments, the one or more nucleic acids encoding the elephant LIF protein comprise a nucleotide sequence selected from the group consisting of SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, and combinations thereof. In certain embodiments, the one or more elephant LIF proteins comprise an amino acid sequence selected from the group consisting of SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NOs:134-140, and combinations thereof.

In certain embodiments, the subject in need thereof is administered (a) one, two, three, four, five, six, seven, eight, nine, or ten nucleic acids encoding an elephant LIF protein; or (b) one, two, three, four, five, six, seven, eight, nine, or ten elephant LIF proteins.

In certain embodiments, the one or more nucleic acids or the one or more proteins are in the form of a composition, wherein the composition further comprises a pharmaceutically acceptable carrier.

In certain embodiments, the one or more nucleic acids encoding an elephant p53 or an elephant LIF are comprised within a vector. The vector can, for example, be delivered to the subject in a composition, wherein the composition further comprises a pharmaceutically acceptable carrier.

In certain embodiments, the subject in need thereof is a mammal. The mammal can, for example, be a human.

The cancer can, for example, be selected from but not limited to, a lung cancer, a gastric cancer, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, and other solid tumors, and a non-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

In another general aspect, the invention relates to a method of inducing apoptosis and/or necrosis in a cancer cell. The methods comprise contacting the cancer cell with (a) one or more nucleic acids encoding an elephant p53 protein, wherein the one or more nucleic acids encoding the elephant p53 protein comprise a nucleotide sequence having at least 80%, at least 85%, at least 90%, at least 91%, at least 92%, at least 93%, at least 94%, at least 95%, at least 96%, at least 97%, at least 98%, or at least 99% identity with a nucleotide sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, and combinations thereof; or (b) one or more elephant p53 proteins, wherein the one or more elephant p53 proteins comprise an amino acid sequence with at least 85%, at least 90%, at least 91%, at least 92%, at least 93%, at least 94%, at least 95%, at least 96%, at least 97%, at least 98%, or at least 99% identity to an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NOs:75-133, and combinations thereof.

In certain embodiments, the one or more nucleic acids encoding the elephant p53 protein comprise a nucleotide sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, and combinations thereof. In certain embodiments, the one or more elephant p53 proteins comprise an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NOs:75-133, and combinations thereof.

In certain embodiments, the cancer cell is contacted with (a) one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, or twenty-five nucleic acids encoding an elephant p53 protein; or (b) one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, thirty-six, thirty-seven, thirty-eight, thirty-nine, forty, forty-one, forty-two, forty-three, forty-four, forty-five, forty-six, forty-seven, forty-eight, forty-nine, or fifty elephant p53 proteins.

In certain embodiments, the cancer cell is further contacted with (c) one or more nucleic acids encoding an elephant leukemia inhibitory factor (LIF) protein, wherein the one or more nucleic acids encoding the elephant LIF protein comprise a nucleotide sequence having at least 80%, at least 85%, at least 90%, at least 91%, at least 92%, at least 93%, at least 94%, at least 95%, at least 96%, at least 97%, at least 98%, or at least 99% identity with a nucleotide sequence selected from the group consisting of SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, and combinations thereof; or (d) one or more elephant LIF proteins, wherein the one or more elephant LIF proteins comprise an amino acid sequence with at least 85%, at least 90%, at least 91%, at least 92%, at least 93%, at least 94%, at least 95%, at least 96%, at least 97%, at least 98%, or at least 99% identity to an amino acid sequence selected from the group consisting of SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NOs:134-140, and combinations thereof

In certain embodiments, the one or more nucleic acids encoding the elephant LIF protein comprise a nucleotide sequence selected from the group consisting of SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, and combinations thereof. In certain embodiments, the one or more elephant LIF proteins comprise an amino acid sequence selected from the group consisting of SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NOs:134-140, and combinations thereof.

In certain embodiments, the cancer cell is contacted with (c) one, two, three, four, five, six, seven, eight, nine, ten, eleven, or twelve nucleic acids encoding an elephant LIF protein; or (d) one, two, three, four, five, six, seven, eight, nine, ten, eleven, twelve, thirteen, fourteen, fifteen, sixteen, seventeen, eighteen, or nineteen elephant LIF proteins.

In another general aspect, the invention relates to a method of inducing apoptosis and/or necrosis in a cancer cell. The methods comprise contacting the cancer cell with (a) one or more nucleic acids encoding an elephant leukemia inhibitory factor (LIF) protein, wherein the one or more nucleic acids encoding the elephant LIF protein comprise a nucleotide sequence having at least 80% identity with a nucleotide sequence selected from the group consisting of SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, and combinations thereof; or (b) one or more elephant LIF proteins, wherein the one or more elephant LIF proteins comprise an amino acid sequence with at least 85% identity to an amino acid sequence selected from the group consisting of SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NOs:134-140, and combinations thereof.

In certain embodiments, the one or more nucleic acids encoding the elephant LIF protein comprise a nucleotide sequence selected from the group consisting of SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, and combinations thereof. In certain embodiments, the one or more elephant LIF proteins comprise an amino acid sequence selected from the group consisting of SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NOs:134-140, and combinations thereof.

In certain embodiments, the cancer cell is contacted with (a) one, two, three, four, five, six, seven, eight, nine, or ten nucleic acids encoding an elephant LIF protein; or (b) one, two, three, four, five, six, seven, eight, nine, or ten elephant LIF proteins.

In certain embodiments, the one or more nucleic acids or the one or more proteins are in the form of a composition, wherein the composition further comprises a pharmaceutically acceptable carrier.

In certain embodiments, the one or more nucleic acids encoding an elephant p53 or an elephant LIF are comprised within a vector. The vector can, for example, be contacted with the cancer cell in a composition, wherein the composition further comprises a pharmaceutically acceptable carrier.

In certain embodiments, the cancer cell is from a mammal. The mammal can, for example, be a human.

The cancer cell can, for example, be a cell from a tumor or cancer selected from, but not limited to, a lung cancer, a gastric cancer, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, and other solid tumors, and a non-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

According to embodiments of the invention, the pharmaceutical compositions comprise a therapeutically effective amount of the nucleic acids and proteins of the invention. As used herein, the term “therapeutically effective amount” refers to an amount of an active ingredient or component that elicits the desired biological or medicinal response in a subject. A therapeutically effective amount can be determined empirically and in a routine manner, in relation to the stated purpose.

As used herein with reference to the nucleic acids and/or proteins of the invention, a therapeutically effective amount means an amount of the nucleic acids and/or proteins that modulate an immune response in a subject in need thereof. Also, as used herein with reference to the nucleic acids and/or proteins, a therapeutically effective amount means an amount of the nucleic acids and/or proteins that results in treatment of a disease, disorder, or condition; prevents or slows the progression of the disease, disorder, or condition; or reduces or completely alleviates symptoms associated with the disease, disorder, or condition.

According to particular embodiments, the disease, disorder or condition to be treated is cancer, preferably a cancer selected from the group consisting of a lung cancer, a gastric cancer, an esophageal cancer, a bile duct cancer, a cholangiocarcinoma, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, and other solid tumors, and a non-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

According to particular embodiments, a therapeutically effective amount refers to the amount of therapy which is sufficient to achieve one, two, three, four, or more of the following effects: (i) reduce or ameliorate the severity of the disease, disorder or condition to be treated or a symptom associated therewith; (ii) reduce the duration of the disease, disorder or condition to be treated, or a symptom associated therewith; (iii) prevent the progression of the disease, disorder or condition to be treated, or a symptom associated therewith; (iv) cause regression of the disease, disorder or condition to be treated, or a symptom associated therewith; (v) prevent the development or onset of the disease, disorder or condition to be treated, or a symptom associated therewith; (vi) prevent the recurrence of the disease, disorder or condition to be treated, or a symptom associated therewith; (vii) reduce hospitalization of a subject having the disease, disorder or condition to be treated, or a symptom associated therewith; (viii) reduce hospitalization length of a subject having the disease, disorder or condition to be treated, or a symptom associated therewith; (ix) increase the survival of a subject with the disease, disorder or condition to be treated, or a symptom associated therewith; (xi) inhibit or reduce the disease, disorder or condition to be treated, or a symptom associated therewith in a subject; and/or (xii) enhance or improve the prophylactic or therapeutic effect(s) of another therapy.

The therapeutically effective amount or dosage can vary according to various factors, such as the disease, disorder or condition to be treated, the means of administration, the target site, the physiological state of the subject (including, e.g., age, body weight, health), whether the subject is a human or an animal, other medications administered, and whether the treatment is prophylactic or therapeutic. Treatment dosages are optimally titrated to optimize safety and efficacy.

According to particular embodiments, the compositions described herein are formulated to be suitable for the intended route of administration to a subject. For example, the compositions described herein can be formulated to be suitable for intravenous, subcutaneous, or intramuscular administration.

As used herein, the terms “treat,” “treating,” and “treatment” are all intended to refer to an amelioration or reversal of at least one measurable physical parameter related to a cancer, which is not necessarily discernible in the subject, but can be discernible in the subject. The terms “treat,” “treating,” and “treatment,” can also refer to causing regression, preventing the progression, or at least slowing down the progression of the disease, disorder, or condition. In a particular embodiment, “treat,” “treating,” and “treatment” refer to an alleviation, prevention of the development or onset, or reduction in the duration of one or more symptoms associated with the disease, disorder, or condition, such as a tumor or more preferably a cancer. In a particular embodiment, “treat,” “treating,” and “treatment” refer to prevention of the recurrence of the disease, disorder, or condition. In a particular embodiment, “treat,” “treating,” and “treatment” refer to an increase in the survival of a subject having the disease, disorder, or condition. In a particular embodiment, “treat,” “treating,” and “treatment” refer to elimination of the disease, disorder, or condition in the subject.

According to particular embodiments, provided are compositions used in the treatment of a cancer. For cancer therapy, the provided compositions can be used in combination with another treatment including, but not limited to, a chemotherapy, an antibody treatment (e.g., anti-PD-1 mAb, anti-PD-L1 mAb, anti-CTLA4 mAb, anti-CD33 mAb, anti-HER-2 mAb, anti-EGFR mAb, and combinations thereof), other immuno-oncology drugs, an antiangiogenic agent, a radiation therapy, an antibody-drug conjugate (ADC), a targeted therapy, or other anticancer drugs.

As used herein, the term “in combination,” in the context of the administration of two or more therapies to a subject, refers to the use of more than one therapy. The use of the term “in combination” does not restrict the order in which therapies are administered to a subject. For example, a first therapy (e.g., a composition described herein) can be administered prior to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject.

### Pharmaceutical Compositions

In another general aspect, the invention relates to a pharmaceutical composition comprising one or more nucleic acids encoding an elephant p53 protein or one or more elephant p53 proteins of the invention and a pharmaceutically acceptable carrier. In another general aspect, the invention relates to a pharmaceutical composition comprising one or more nucleic acids encoding an elephant LIF protein or one or more elephant LIF proteins of the invention and a pharmaceutically acceptable carrier. In certain embodiments, the invention relates to a pharmaceutical composition comprising one or more nucleic acids encoding an elephant p53 protein or one or more elephant p53 proteins and further comprising one or more nucleic acids encoding an elephant LIF protein or one or more elephant LIF proteins of the invention and a pharmaceutically acceptable carrier.

The term “pharmaceutical composition” as used herein means a product comprising an isolated nucleic acid of the invention and/or an isolated protein of the invention together with a pharmaceutically acceptable carrier. Nucleic acids and proteins of the invention and compositions comprising them are also useful in the manufacture of a medicament for therapeutic applications mentioned herein.

In certain embodiments, provided are pharmaceutical compositions comprise a pharmaceutically acceptable carrier and (a) one or more nucleic acids encoding an elephant p53 protein, wherein the one or more nucleic acids encoding the elephant p53 protein comprise a nucleotide sequence having at least 80%, at least 85%, at least 90%, at least 91%, at least 92%, at least 93%, at least 94%, at least 95%, at least 96%, at least 97%, at least 98%, or at least 99% identity with a nucleotide sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, and combinations thereof; or (b) one or more elephant p53 proteins, wherein the one or more elephant p53 proteins comprise an amino acid sequence with at least 85%, at least 90%, at least 91%, at least 92%, at least 93%, at least 94%, at least 95%, at least 96%, at least 97%, at least 98%, or at least 99% identity to an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NOs:75-133, and combinations thereof.

In certain embodiments, the one or more nucleic acids encoding the elephant p53 protein comprise a nucleotide sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, and combinations thereof. In certain embodiments, the one or more elephant p53 proteins comprise an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NOs:75-133, and combinations thereof.

In certain embodiments, the pharmaceutical composition comprises (a) one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, or twenty-five nucleic acids encoding an elephant p53 protein; or (b) one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, thirty-six, thirty-seven, thirty-eight, thirty-nine, forty, forty-one, forty-two, forty-three, forty-four, forty-five, forty-six, forty-seven, forty-eight, forty-nine, or fifty elephant p53 proteins.

In certain embodiments, the pharmaceutical composition further comprises (c) one or more nucleic acids encoding an elephant leukemia inhibitory factor (LIF) protein, wherein the one or more nucleic acids encoding the elephant LIF protein comprise a nucleotide sequence having at least 80%, at least 85%, at least 90%, at least 91%, at least 92%, at least 93%, at least 94%, at least 95%, at least 96%, at least 97%, at least 98%, or at least 99% identity with a nucleotide sequence selected from the group consisting of SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, and combinations thereof; or (d) one or more elephant LIF proteins, wherein the one or more elephant LIF proteins comprise an amino acid sequence with at least 85%, at least 90%, at least 91%, at least 92%, at least 93%, at least 94%, at least 95%, at least 96%, at least 97%, at least 98%, or at least 99% identity to an amino acid sequence selected from the group consisting of SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NOs:134-140, and combinations thereof

In certain embodiments, the one or more nucleic acids encoding the elephant LIF protein comprise a nucleotide sequence selected from the group consisting of SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, and combinations thereof. In certain embodiments, the one or more elephant LIF proteins comprise an amino acid sequence selected from the group consisting of SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NOs:134-140, and combinations thereof.

In certain embodiments, the pharmaceutical composition comprises (c) one, two, three, four, five, six, seven, eight, nine, ten, eleven, or twelve nucleic acids encoding an elephant LIF protein; or (d) one, two, three, four, five, six, seven, eight, nine, ten, eleven, twelve, thirteen, fourteen, fifteen, sixteen, seventeen, eighteen, or nineteen elephant LIF proteins.

In certain embodiments, the pharmaceutical compositions comprise a pharmaceutically acceptable carrier and (a) one or more nucleic acids encoding an elephant leukemia inhibitory factor (LIF) protein, wherein the one or more nucleic acids encoding the elephant LIF protein comprise a nucleotide sequence having at least 80% identity with a nucleotide sequence selected from the group consisting of SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, and combinations thereof; or (b) one or more elephant LIF proteins, wherein the one or more elephant LIF proteins comprise an amino acid sequence with at least 85% identity to an amino acid sequence selected from the group consisting of SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NOs:134-140, and combinations thereof.

In certain embodiments, the one or more nucleic acids encoding the elephant LIF protein comprise a nucleotide sequence selected from the group consisting of SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, and combinations thereof. In certain embodiments, the one or more elephant LIF proteins comprise an amino acid sequence selected from the group consisting of SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NOs:134-140, and combinations thereof.

In certain embodiments, the pharmaceutical composition comprises (a) one, two, three, four, five, six, seven, eight, nine, ten, eleven, or twelve nucleic acids encoding an elephant LIF protein; or (b) one, two, three, four, five, six, seven, eight, nine, ten, eleven, twelve, thirteen, fourteen, fifteen, sixteen, seventeen, eighteen, or nineteen elephant LIF proteins.

As used herein, the term “carrier” refers to any excipient, diluent, filler, salt, buffer, stabilizer, solubilizer, oil, lipid, lipid containing vesicle, microsphere, liposomal encapsulation, or other material well known in the art for use in pharmaceutical formulations. It will be understood that the characteristics of the carrier, excipient or diluent will depend on the route of administration for a particular application. As used herein, the term “pharmaceutically acceptable carrier” refers to a non-toxic material that does not interfere with the effectiveness of a composition according to the invention or the biological activity of a composition according to the invention. According to particular embodiments, in view of the present disclosure, any pharmaceutically acceptable carrier suitable for use in a pharmaceutical composition comprising one or more nucleic acids and/or one or more proteins can be used in the invention.

The formulation of pharmaceutically active ingredients with pharmaceutically acceptable carriers is known in the art, e.g., Remington: The Science and Practice of Pharmacy (e.g., 21st edition (2005), and any later editions). Non-limiting examples of additional ingredients include buffers, diluents, solvents, tonicity regulating agents, preservatives, stabilizers, and chelating agents. One or more pharmaceutically acceptable carrier(s) can be used in formulating the pharmaceutical compositions of the invention.

In one embodiment of the invention, the pharmaceutical composition is a liquid formulation. A preferred example of a liquid formulation is an aqueous formulation, i.e., a formulation comprising water. The liquid formulation can comprise a solution, a suspension, an emulsion, a microemulsion, a gel, and the like. An aqueous formulation typically comprises at least 50% w/w water, or at least 60%, 70%, 75%, 80%, 85%, 90%, or at least 95% w/w of water.

In one embodiment, the pharmaceutical composition can be formulated as an injectable which can be injected, for example, via an injection device (e.g., a syringe or an infusion pump). The injection can be delivered subcutaneously, intramuscularly, intraperitoneally, intravitreally, or intravenously, for example.

In another embodiment, the pharmaceutical composition is a solid formulation, e.g., a freeze-dried or spray-dried composition, which can be used as is, or whereto the physician or the patient adds solvents, and/or diluents prior to use. Solid dosage forms can include tablets, such as compressed tablets, and/or coated tablets, and capsules (e.g., hard or soft gelatin capsules). The pharmaceutical composition can also be in the form of sachets, dragees, powders, granules, lozenges, or powders for reconstitution, for example.

The dosage forms may be immediate release, in which case they can comprise a water-soluble or dispersible carrier, or they can be delayed release, sustained release, or modified release, in which case they can comprise water-insoluble polymers that regulate the rate of dissolution of the dosage form in the gastrointestinal tract or under the skin.

In other embodiments, the pharmaceutical composition can be delivered intranasally, intrabuccally, or sublingually.

The pH in an aqueous formulation can be between pH 3 and pH 10. In one embodiment of the invention, the pH of the formulation is from about 7.0 to about 9.5. In another embodiment of the invention, the pH of the formulation is from about 3.0 to about 7.0.

In another embodiment of the invention, the pharmaceutical composition comprises a buffer. Non-limiting examples of buffers include: arginine, aspartic acid, bicine, citrate, disodium hydrogen phosphate, fumaric acid, glycine, glycylglycine, histidine, lysine, maleic acid, malic acid, sodium acetate, sodium carbonate, sodium dihydrogen phosphate, sodium phosphate, succinate, tartaric acid, tricine, and tris(hydroxymethyl)-aminomethane, and mixtures thereof. The buffer can be present individually or in the aggregate, in a concentration from about 0.01 mg/ml to about 50 mg/ml, for example from about 0.1 mg/ml to about 20 mg/ml. Pharmaceutical compositions comprising each one of these specific buffers constitute alternative embodiments of the invention.

In another embodiment of the invention, the pharmaceutical composition comprises a preservative. Non-limiting examples of preservatives include: benzethonium chloride, benzoic acid, benzyl alcohol, bronopol, butyl 4-hydroxybenzoate, chlorobutanol, chlorocresol, chlorohexidine, chlorphenesin, o-cresol, m-cresol, p-cresol, ethyl 4-hydroxybenzoate, imidurea, methyl 4-hydroxybenzoate, phenol, 2-phenoxyethanol, 2-phenylethanol, propyl 4-hydroxybenzoate, sodium dehydroacetate, thiomerosal, and mixtures thereof. The preservative can be present individually or in the aggregate, in a concentration from about 0.01 mg/ml to about 50 mg/ml, for example from about 0.1 mg/ml to about 20 mg/ml. Pharmaceutical compositions comprising each one of these specific preservatives constitute alternative embodiments of the invention.

In another embodiment of the invention, the pharmaceutical composition comprises an isotonic agent. Non-limiting examples of isotonic agents include a salt (such as sodium chloride), an amino acid (such as glycine, histidine, arginine, lysine, isoleucine, aspartic acid, tryptophan, and threonine), an alditol (such as glycerol, 1,2-propanediol propyleneglycol), 1,3-propanediol, and 1,3-butanediol), polyethyleneglycol (e.g., PEG400), and mixtures thereof. Another example of an isotonic agent includes a sugar. Non-limiting examples of sugars may be mono-, di-, or polysaccharides, or water-soluble glucans, including for example fructose, glucose, mannose, sorbose, xylose, maltose, lactose, sucrose, trehalose, dextran, pullulan, dextrin, cyclodextrin, alpha and beta-HPCD, soluble starch, hydroxyethyl starch, and sodium carboxymethylcellulose. Another example of an isotonic agent is a sugar alcohol, wherein the term “sugar alcohol” is defined as a C(4-8) hydrocarbon having at least one-OH group. Non-limiting examples of sugar alcohols include mannitol, sorbitol, inositol, galactitol, dulcitol, xylitol, and arabitol. The isotonic agent can be present individually or in the aggregate, in a concentration from about 0.01 mg/ml to about 50 mg/ml, for example from about 0.1 mg/ml to about 20 mg/ml.

Pharmaceutical compositions comprising each one of these specific isotonic agents constitute alternative embodiments of the invention.

In another embodiment of the invention, the pharmaceutical composition comprises a chelating agent. Non-limiting examples of chelating agents include citric acid, aspartic acid, salts of ethylenediaminetetraacetic acid (EDTA), and mixtures thereof. The chelating agent can be present individually or in the aggregate, in a concentration from about 0.01 mg/ml to about 50 mg/ml, for example from about 0.1 mg/ml to about 20 mg/ml. Pharmaceutical compositions comprising each one of these specific chelating agents constitute alternative embodiments of the invention.

In another embodiment of the invention, the pharmaceutical composition comprises a stabilizer. Non-limiting examples of stabilizers include one or more aggregation inhibitors, one or more oxidation inhibitors, one or more surfactants, and/or one or more protease inhibitors.

In another embodiment of the invention, the pharmaceutical composition comprises a stabilizer, wherein said stabilizer is carboxy-/hydroxycellulose and derivatives thereof (such as HPC, HPC-SL, HPC-L and HPMC), cyclodextrins, 2-methylthioethanol, polyethylene glycol (such as PEG 3350), polyvinyl alcohol (PVA), polyvinyl pyrrolidone, salts (such as sodium chloride), sulphur-containing substances such as monothioglycerol), or thioglycolic acid. The stabilizer can be present individually or in the aggregate, in a concentration from about 0.01 mg/ml to about 50 mg/ml, for example from about 0.1 mg/ml to about 20 mg/ml. Pharmaceutical compositions comprising each one of these specific stabilizers constitute alternative embodiments of the invention.

In further embodiments of the invention, the pharmaceutical composition comprises one or more surfactants, preferably a surfactant, at least one surfactant, or two different surfactants. The term “surfactant” refers to any molecules or ions that are comprised of a water-soluble (hydrophilic) part, and a fat-soluble (lipophilic) part. The surfactant can, for example, be selected from the group consisting of anionic surfactants, cationic surfactants, nonionic surfactants, and/or zwitterionic surfactants. The surfactant can be present individually or in the aggregate, in a concentration from about 0.1 mg/ml to about 20 mg/ml. Pharmaceutical compositions comprising each one of these specific surfactants constitute alternative embodiments of the invention.

In a further embodiment of the invention, the pharmaceutical composition comprises one or more protease inhibitors, such as, e.g., EDTA, and/or benzamidine hydrochloric acid (HCl). The protease inhibitor can be present individually or in the aggregate, in a concentration from about 0.1 mg/ml to about 20 mg/ml. Pharmaceutical compositions comprising each one of these specific protease inhibitors constitute alternative embodiments of the invention.

In another general aspect, the invention relates to a method of producing a pharmaceutical composition comprising a one or more nucleic acids or one or more proteins of the invention, comprising combining the one or more nucleic acids or one or more proteins with a pharmaceutically acceptable carrier to obtain the pharmaceutical composition.

### Kits

Also provided are kits for treating or preventing cancer in a subject in need thereof. The kits comprise (a) one or more nucleic acids encoding an elephant p53 protein, wherein the one or more nucleic acids encoding the elephant p53 protein comprise a nucleotide sequence having at least 80%, at least 85%, at least 90%, at least 91%, at least 92%, at least 93%, at least 94%, at least 95%, at least 96%, at least 97%, at least 98%, or at least 99% identity with a nucleotide sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, and combinations thereof; or (b) one or more elephant p53 proteins, wherein the one or more elephant p53 proteins comprise an amino acid sequence with at least 85%, at least 90%, at least 91%, at least 92%, at least 93%, at least 94%, at least 95%, at least 96%, at least 97%, at least 98%, or at least 99% identity to an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NOs:75-133, and combinations thereof; and (c) instructions for use.

In certain embodiments, the one or more nucleic acids encoding the elephant p53 protein comprise a nucleotide sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, and combinations thereof. In certain embodiments, the one or more elephant p53 proteins comprise an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ SEQ ID NO:50, SEQ ID NOs:75-133, and combinations thereof.

In certain embodiments, the kit comprises (a) one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, or twenty-five nucleic acids encoding an elephant p53 protein; or (b) one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, thirty-six, thirty-seven, thirty-eight, thirty-nine, forty, forty-one, forty-two, forty-three, forty-four, forty-five, forty-six, forty-seven, forty-eight, forty-nine, or fifty elephant p53 proteins.

In certain embodiments, the kit further comprises (d) one or more nucleic acids encoding an elephant leukemia inhibitory factor (LIF) protein, wherein the one or more nucleic acids encoding the elephant LIF protein comprise a nucleotide sequence having at least 80%, at least 85%, at least 90%, at least 91%, at least 92%, at least 93%, at least 94%, at least 95%, at least 96%, at least 97%, at least 98%, or at least 99% identity with a nucleotide sequence selected from the group consisting of SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, and combinations thereof; or (e) one or more elephant LIF proteins, wherein the one or more elephant LIF proteins comprise an amino acid sequence with at least 85%, at least 90%, at least 91%, at least 92%, at least 93%, at least 94%, at least 95%, at least 96%, at least 97%, at least 98%, or at least 99% identity to an amino acid sequence selected from the group consisting of SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NOs:134-140, and combinations thereof.

In certain embodiments, the one or more nucleic acids encoding the elephant LIF protein comprise a nucleotide sequence selected from the group consisting of SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, and combinations thereof. In certain embodiments, the one or more elephant LIF proteins comprise an amino acid sequence selected from the group consisting of SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NOs:134-140, and combinations thereof.

In certain embodiments, the kit comprises (d) one, two, three, four, five, six, seven, eight, nine, ten, eleven, or twelve nucleic acids encoding an elephant LIF protein; or (e) one, two, three, four, five, six, seven, eight, nine, ten, eleven, twelve, thirteen, fourteen, fifteen, sixteen, seventeen, eighteen, or nineteen elephant LIF proteins.

In certain embodiments, the kits comprise (a) one or more nucleic acids encoding an elephant leukemia inhibitory factor (LIF) protein, wherein the one or more nucleic acids encoding the elephant LIF protein comprise a nucleotide sequence having at least 80%, at least 85%, at least 90%, at least 91%, at least 92%, at least 93%, at least 94%, at least 95%, at least 96%, at least 97%, at least 98%, or at least 99% identity with a nucleotide sequence selected from the group consisting of SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, and combinations thereof; or (b) one or more elephant LIF proteins, wherein the one or more elephant LIF proteins comprise an amino acid sequence with at least 85%, at least 90%, at least 91%, at least 92%, at least 93%, at least 94%, at least 95%, at least 96%, at least 97%, at least 98%, or at least 99% identity to an amino acid sequence selected from the group consisting of SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NOs:134-140, and combinations thereof; and (c) instructions for use.

In certain embodiments, the one or more nucleic acids encoding the elephant LIF protein comprise a nucleotide sequence selected from the group consisting of SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, and combinations thereof. In certain embodiments, the one or more elephant LIF proteins comprise an amino acid sequence selected from the group consisting of SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NOs:134-140, and combinations thereof.

In certain embodiments, the kit comprises (a) one, two, three, four, five, six, seven, eight, nine, or ten nucleic acids encoding an elephant LIF protein; or (b) one, two, three, four, five, six, seven, eight, nine, or ten elephant LIF proteins.

In certain embodiments, the one or more nucleic acids or the one or more proteins are in the form of a composition, and wherein the composition further comprises a pharmaceutically acceptable carrier.

In certain embodiments, the subject in need thereof is a mammal. The mammal can, for example, be a human.

In certain embodiments, the cancer is selected from a lung cancer, a gastric cancer, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, and other solid tumors, and a non-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

### EMBODIMENTS

The invention provides also the following non-limiting embodiments.

Embodiment 1 is a method of treating or preventing cancer in a subject in need thereof, the method comprising administering to the subject in need thereof:
(a) one or more nucleic acids encoding an elephant p53 protein, wherein the one or more nucleic acids encoding the elephant p53 protein comprise a nucleotide sequence having at least 80%, at least 85%, at least 90%, at least 91%, at least 92%, at least 93%, at least 94%, at least 95%, at least 96%, at least 97%, at least 98%, or at least 99% identity with a nucleotide sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, and combinations thereof; or
(b) one or more elephant p53 proteins, wherein the one or more elephant p53 proteins comprise an amino acid sequence with at least 85%, at least 90%, at least 91%, at least 92%, at least 93%, at least 94%, at least 95%, at least 96%, at least 97%, at least 98%, or at least 99% identity to an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NOs:75-133, and combinations thereof.

Embodiment 2 is the method of embodiment 1, wherein the one or more nucleic acids encoding the elephant p53 protein comprise a nucleotide sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, and combinations thereof.

Embodiment 3 is the method of embodiment 1 or 2, wherein the one or more elephant p53 proteins comprise an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NOs:75-133, and combinations thereof.

Embodiment 4 is the method of any one of embodiments 1-3, wherein the subject in need thereof is administered:
(a) one, two, three, four, five, six, seven, eight, nine, or ten nucleic acids encoding an elephant p53 protein; or
(b) one, two, three, four, five six, seven, eight, nine, or ten elephant p53 proteins.

Embodiment 5 is the method of any one of embodiments 1-4, wherein the subject in need thereof is further administered:
(c) one or more nucleic acids encoding an elephant leukemia inhibitory factor (LIF) protein, wherein the one or more nucleic acids encoding the elephant LIF protein comprise a nucleotide sequence having at least 80%, at least 85%, at least 90%, at least 91%, at least 92%, at least 93%, at least 94%, at least 95%, at least 96%, at least 97%, at least 98%, or at least 99% identity with a nucleotide sequence selected from the group consisting of SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, and combinations thereof; or
(d) one or more elephant LIF proteins, wherein the one or more elephant LIF proteins comprise an amino acid sequence with at least 85%, at least 90%, at least 91%, at least 92%, at least 93%, at least 94%, at least 95%, at least 96%, at least 97%, at least 98%, or at least 99% identity to an amino acid sequence selected from the group consisting of SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NOs:134-140, and combinations thereof.

Embodiment 6 is the method of embodiment 5, wherein the one or more nucleic acids encoding the elephant LIF protein comprise a nucleotide sequence selected from the group consisting of SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, and combinations thereof.

Embodiment 7 is the method of embodiment 5 or 6, wherein the one or more elephant LIF proteins comprise an amino acid sequence selected from the group consisting of SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NOs:134-140, and combinations thereof.

Embodiment 8 is the method of any one of embodiments 5-7, wherein the subject in need thereof is administered:
(c) one, two, three, four, five, six, seven, eight, nine, or ten nucleic acids encoding an elephant LIF protein; or
(d) one, two, three, four, five, six, seven, eight, nine, or ten elephant LIF proteins.

Embodiment 9 is the method of any one of embodiments 1-8, wherein the one or more nucleic acids or the one or more proteins are in the form of a composition, wherein the composition further comprises a pharmaceutically acceptable carrier.

Embodiment 10 is the method of any one of embodiments 1-9, wherein the subject in need thereof is a mammal.

Embodiment 11 is the method of embodiment 10, wherein the mammal is a human.

Embodiment 12 is the method of any one of embodiments 1-11, wherein the cancer is selected from a lung cancer, a gastric cancer, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, and other solid tumors, and a non-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

Embodiment 13 is a pharmaceutical composition comprising a pharmaceutically acceptable carrier and:
(a) one or more nucleic acids encoding an elephant p53 protein, wherein the one or more nucleic acids encoding the elephant p53 protein comprise a nucleotide sequence having at least 80%, at least 85%, at least 90%, at least 91%, at least 92%, at least 93%, at least 94%, at least 95%, at least 96%, at least 97%, at least 98%, or at least 99% identity with a nucleotide sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, and combinations thereof; or
(b) one or more elephant p53 proteins, wherein the one or more elephant p53 proteins comprise an amino acid sequence with at least 85%, at least 90%, at least 91%, at least 92%, at least 93%, at least 94%, at least 95%, at least 96%, at least 97%, at least 98%, or at least 99% identity to an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NOs:75-133, and combinations thereof.

Embodiment 14 is the pharmaceutical composition of embodiment 13, wherein the one or more nucleic acids encoding the elephant p53 protein comprise a nucleotide sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, and combinations thereof.

Embodiment 15 is the pharmaceutical composition of embodiment 13 or 14, wherein the one or more elephant p53 proteins comprise an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NOs:75-133, and combinations thereof.

Embodiment 16 is the pharmaceutical composition of any one of embodiments 13-15, wherein the composition comprises:
(a) one, two, three, four, five, six, seven, eight, nine, or ten nucleic acids encoding an elephant p53 protein; or
(b) one, two, three, four, five six, seven, eight, nine, or ten elephant p53 proteins.

Embodiment 17 is the pharmaceutical composition of any one of embodiments 13-16, wherein the pharmaceutical composition further comprises:
(c) one or more nucleic acids encoding an elephant leukemia inhibitory factor (LIF) protein, wherein the one or more nucleic acids encoding the elephant LIF protein comprise a nucleotide sequence having at least 80%, at least 85%, at least 90%, at least 91%, at least 92%, at least 93%, at least 94%, at least 95%, at least 96%, at least 97%, at least 98%, or at least 99% identity with a nucleotide sequence selected from the group consisting of SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, and combinations thereof; or
(d) one or more elephant LIF proteins, wherein the one or more elephant LIF proteins comprise an amino acid sequence with at least 85%, at least 90%, at least 91%, at least 92%, at least 93%, at least 94%, at least 95%, at least 96%, at least 97%, at least 98%, or at least 99% identity to an amino acid sequence selected from the group consisting of SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NOs:134-140, and combinations thereof.

Embodiment 18 is the pharmaceutical composition of embodiment 17, wherein the one or more nucleic acids encoding the elephant LIF protein comprise a nucleotide sequence selected from the group consisting of SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, and combinations thereof.

Embodiment 19 is the pharmaceutical composition of embodiment 17 or 18, wherein the one or more elephant LIF proteins comprise an amino acid sequence selected from the group consisting of SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NOs:134-140, and combinations thereof.

Embodiment 20 is the pharmaceutical composition of any one of embodiments 17-19, wherein the pharmaceutical composition comprises:
(c) one, two, three, four, five, six, seven, eight, nine, or ten nucleic acids encoding an elephant LIF protein; or
(d) one, two, three, four, five, six, seven, eight, nine, or ten elephant LIF proteins.

Embodiment 21 is a kit for treating or preventing cancer in a subject in need thereof, the kit comprising:
(a) one or more nucleic acids encoding an elephant p53 protein, wherein the one or more nucleic acids encoding the elephant p53 protein comprise a nucleotide sequence having at least 80%, at least 85%, at least 90%, at least 91%, at least 92%, at least 93%, at least 94%, at least 95%, at least 96%, at least 97%, at least 98%, or at least 99% identity with a nucleotide sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, and combinations thereof; or
(b) one or more elephant p53 proteins, wherein the one or more elephant p53 proteins comprise an amino acid sequence with at least 85%, at least 90%, at least 91%, at least 92%, at least 93%, at least 94%, at least 95%, at least 96%, at least 97%, at least 98%, or at least 99% identity to an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NOs:75-133, and combinations thereof, and
(c) instructions for use.

Embodiment 22 is the kit of embodiment 21, wherein the one or more nucleic acids encoding the elephant p53 protein comprise a nucleotide sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, and combinations thereof.

Embodiment 23 is the kit of embodiment 21 or 22, wherein the one or more elephant p53 proteins comprise an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NOs:75-133, and combinations thereof.

Embodiment 24 is the kit of any one of embodiments 21-23, wherein the kit comprises:
(a) one, two, three, four, five, six, seven, eight, nine, or ten nucleic acids encoding an elephant p53 protein; or
(b) one, two, three, four, five six, seven, eight, nine, or ten elephant p53 proteins.

Embodiment 25 is the kit of any one of embodiments 21-24, wherein the kit further comprises:
(d) one or more nucleic acids encoding an elephant leukemia inhibitory factor (LIF) protein, wherein the one or more nucleic acids encoding the elephant LIF protein comprise a nucleotide sequence having at least 80%, at least 85%, at least 90%, at least 91%, at least 92%, at least 93%, at least 94%, at least 95%, at least 96%, at least 97%, at least 98%, or at least 99% identity with a nucleotide sequence selected from the group consisting of SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, and combinations thereof; or
(e) one or more elephant LIF proteins, wherein the one or more elephant LIF proteins comprise an amino acid sequence with at least 85%, at least 90%, at least 91%, at least 92%, at least 93%, at least 94%, at least 95%, at least 96%, at least 97%, at least 98%, or at least 99% identity to an amino acid sequence selected from the group consisting of SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NOs:134-140, and combinations thereof.

Embodiment 26 is the kit of embodiment 25, wherein the one or more nucleic acids encoding the elephant LIF protein comprise a nucleotide sequence selected from the group consisting of SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, and combinations thereof.

Embodiment 27 is the kit of embodiment 25 or 26, wherein the one or more elephant LIF proteins comprise an amino acid sequence selected from the group consisting of SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NOs:134-140, and combinations thereof.

Embodiment 28 is the kit of any one of embodiments 25-27, wherein the kit comprises:
(d) one, two, three, four, five, six, seven, eight, nine, or ten nucleic acids encoding an elephant LIF protein; or
(e) one, two, three, four, five, six, seven, eight, nine, or ten elephant LIF proteins.

Embodiment 29 is the kit of any one of embodiments 21-28, wherein the one or more nucleic acids or the one or more proteins are in the form of a composition, and wherein the composition further comprises a pharmaceutically acceptable carrier.

Embodiment 30 is the kit of any one of embodiments 21-29, wherein the subject in need thereof is a mammal.

Embodiment 31 is the method of embodiment 30, wherein the mammal is a human.

Embodiment 32 is the kit of any one of embodiments 21-31, wherein the cancer is selected from a lung cancer, a gastric cancer, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, and other solid tumors, and a non-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

Embodiment 33 is a method of treating or preventing cancer in a subject in need thereof, the method comprising administering to the subject in need thereof:
(a) one or more nucleic acids encoding an elephant leukemia inhibitory factor (LIF) protein, wherein the one or more nucleic acids encoding the elephant LIF protein comprise a nucleotide sequence having at least 80% identity with a nucleotide sequence selected from the group consisting of SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, and combinations thereof; or
(b) one or more elephant LIF proteins, wherein the one or more elephant LIF proteins comprise an amino acid sequence with at least 85% identity to an amino acid sequence selected from the group consisting of SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NOs:134-140, and combinations thereof.

Embodiment 34 is the method of embodiment 33, wherein the one or more nucleic acids encoding the elephant LIF protein comprise a nucleotide sequence selected from the group consisting of SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, and combinations thereof.

Embodiment 35 is the method of embodiment 33 or 34, wherein the one or more elephant LIF proteins comprise an amino acid sequence selected from the group consisting of SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NOs:134-140, and combinations thereof.

Embodiment 36 is the method of any one of embodiments 33-35, wherein the subject in need thereof is administered:
(a) one, two, three, four, five, six, seven, eight, nine, or ten nucleic acids encoding an elephant LIF protein; or
(b) one, two, three, four, five, six, seven, eight, nine, or ten elephant LIF proteins.

Embodiment 37 is the method of any one of embodiments 33-36, wherein the one or more nucleic acids or the one or more proteins are in the form of a composition, wherein the composition further comprises a pharmaceutically acceptable carrier.

Embodiment 38 is the method of any one of embodiments 33-37, wherein the subject in need thereof is a mammal.

Embodiment 39 is the method of embodiment 38, wherein the mammal is a human.

Embodiment 40 is the method of any one of embodiments 33-39, wherein the cancer is selected from a lung cancer, a gastric cancer, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, and other solid tumors, and a non-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

Embodiment 41 is a pharmaceutical composition comprising a pharmaceutically acceptable carrier and:
(a) one or more nucleic acids encoding an elephant leukemia inhibitory factor (LIF) protein, wherein the one or more nucleic acids encoding the elephant LIF protein comprise a nucleotide sequence having at least 80% identity with a nucleotide sequence selected from the group consisting of SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, and combinations thereof; or
(b) one or more elephant LIF proteins, wherein the one or more elephant LIF proteins comprise an amino acid sequence with at least 85% identity to an amino acid sequence selected from the group consisting of SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID

NOs:134-140, and combinations thereof.

Embodiment 42 is the pharmaceutical composition of embodiment 41, wherein the one or more nucleic acids encoding the elephant LIF protein comprise a nucleotide sequence selected from the group consisting of SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, and combinations thereof.

Embodiment 43 is the pharmaceutical composition of embodiment 41 or 42, wherein the one or more elephant LIF proteins comprise an amino acid sequence selected from the group consisting of SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NOs:134-140, and combinations thereof.

Embodiment 44 is the pharmaceutical composition of any one of embodiments 41-43, wherein the pharmaceutical composition comprises:
(a) one, two, three, four, five, six, seven, eight, nine, or ten nucleic acids encoding an elephant LIF protein; or
(b) one, two, three, four, five, six, seven, eight, nine, or ten elephant LIF proteins.

Embodiment 45 is a kit for treating or preventing cancer in a subject in need thereof, the kit comprising:
(a) one or more nucleic acids encoding an elephant leukemia inhibitory factor (LIF) protein, wherein the one or more nucleic acids encoding the elephant LIF protein comprise a nucleotide sequence having at least 80% identity with a nucleotide sequence selected from the group consisting of SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, and combinations thereof; or
(b) one or more elephant LIF proteins, wherein the one or more elephant LIF proteins comprise an amino acid sequence with at least 85% identity to an amino acid sequence selected from the group consisting of SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NOs:134-140, and combinations thereof; and
(c) instructions for use.

Embodiment 46 is the kit of embodiment 45, wherein the one or more nucleic acids encoding the elephant LIF protein comprise a nucleotide sequence selected from the group consisting of SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, and combinations thereof.

Embodiment 47 is the kit of embodiment 45 or 46, wherein the one or more elephant LIF proteins comprise an amino acid sequence selected from the group consisting of SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NOs:134-140, and combinations thereof.

Embodiment 48 is the kit of any one of embodiments 45-47, wherein the kit comprises:
(a) one, two, three, four, five, six, seven, eight, nine, or ten nucleic acids encoding an elephant LIF protein; or
(b) one, two, three, four, five, six, seven, eight, nine, or ten elephant LIF proteins.

Embodiment 49 is the kit of any one of embodiments 45-48, wherein the one or more nucleic acids or the one or more proteins are in the form of a composition, and wherein the composition further comprises a pharmaceutically acceptable carrier.

Embodiment 50 is the kit of any one of embodiments 45-49, wherein the subject in need thereof is a mammal.

Embodiment 51 is the method of embodiment 50, wherein the mammal is a human.

Embodiment 52 is the kit of any one of embodiments 45-51, wherein the cancer is selected from a lung cancer, a gastric cancer, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, and other solid tumors, and a non-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

Embodiment 53 is a method of inducing apoptosis and/or necrosis in a cancer cell, the method comprising contacting the cancer cell with:
(a) one or more nucleic acids encoding an elephant p53 protein, wherein the one or more nucleic acids encoding the elephant p53 protein comprise a nucleotide sequence having at least 80%, at least 85%, at least 90%, at least 91%, at least 92%, at least 93%, at least 94%, at least 95%, at least 96%, at least 97%, at least 98%, or at least 99% identity with a nucleotide sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, and combinations thereof; or
(b) one or more elephant p53 proteins, wherein the one or more elephant p53 proteins comprise an amino acid sequence with at least 85%, at least 90%, at least 91%, at least 92%, at least 93%, at least 94%, at least 95%, at least 96%, at least 97%, at least 98%, or at least 99% identity to an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NOs:75-133, and combinations thereof.

Embodiment 54 is the method of embodiment 53, wherein the one or more nucleic acids encoding the elephant p53 protein comprise a nucleotide sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, and combinations thereof.

Embodiment 55 is the method of embodiment 53 or 54, wherein the one or more elephant p53 proteins comprise an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NOs:75-133, and combinations thereof.

Embodiment 56 is the method of any one of embodiments 53-55, wherein the cancer cell is contacted with:
(a) one, two, three, four, five, six, seven, eight, nine, or ten nucleic acids encoding an elephant p53 protein; or
(b) one, two, three, four, five six, seven, eight, nine, or ten elephant p53 proteins.

Embodiment 57 is the method of any one of embodiments 53-56, wherein the cancer cell is further contacted with:
(c) one or more nucleic acids encoding an elephant leukemia inhibitory factor (LIF) protein, wherein the one or more nucleic acids encoding the elephant LIF protein comprise a nucleotide sequence having at least 80%, at least 85%, at least 90%, at least 91%, at least 92%, at least 93%, at least 94%, at least 95%, at least 96%, at least 97%, at least 98%, or at least 99% identity with a nucleotide sequence selected from the group consisting of SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, and combinations thereof; or
(d) one or more elephant LIF proteins, wherein the one or more elephant LIF proteins comprise an amino acid sequence with at least 85%, at least 90%, at least 91%, at least 92%, at least 93%, at least 94%, at least 95%, at least 96%, at least 97%, at least 98%, or at least 99% identity to an amino acid sequence selected from the group consisting of SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NOs:134-140, and combinations thereof.

Embodiment 58 is the method of embodiment 57, wherein the one or more nucleic acids encoding the elephant LIF protein comprise a nucleotide sequence selected from the group consisting of SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, and combinations thereof.

Embodiment 59 is the method of embodiment 57 or 58, wherein the one or more elephant LIF proteins comprise an amino acid sequence selected from the group consisting of SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NOs:134-140, and combinations thereof.

Embodiment 60 is the method of any one of embodiments 57-59, wherein the cancer cell is contacted with:
(c) one, two, three, four, five, six, seven, eight, nine, or ten nucleic acids encoding an elephant LIF protein; or
(d) one, two, three, four, five, six, seven, eight, nine, or ten elephant LIF proteins.

Embodiment 61 is the method of any one of embodiments 53-60, wherein the one or more nucleic acids or the one or more proteins are in the form of a composition, wherein the composition further comprises a pharmaceutically acceptable carrier.

Embodiment 62 is the method of any one of embodiments 53-61, wherein the cancer cell is from a mammal.

Embodiment 63 is the method of embodiment 62, wherein the mammal is a human.

Embodiment 64 is the method of any one of embodiments 53-63, wherein the cancer cell is from a tumor or cancer selected from a lung cancer, a gastric cancer, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, and other solid tumors, and a non-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

Embodiment 65 is a method of inducing apoptosis and/or necrosis in a cancer cell, the method comprising contacting the cancer cell with:
(a) one or more nucleic acids encoding an elephant leukemia inhibitory factor (LIF) protein, wherein the one or more nucleic acids encoding the elephant LIF protein comprise a nucleotide sequence having at least 80% identity with a nucleotide sequence selected from the group consisting of SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, and combinations thereof; or
(b) one or more elephant LIF proteins, wherein the one or more elephant LIF proteins comprise an amino acid sequence with at least 85% identity to an amino acid sequence selected from the group consisting of SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NOs:134-140, and combinations thereof.

Embodiment 66 is the method of embodiment 65, wherein the one or more nucleic acids encoding the elephant LIF protein comprise a nucleotide sequence selected from the group consisting of SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, and combinations thereof.

Embodiment 67 is the method of embodiment 65 or 66, wherein the one or more elephant LIF proteins comprise an amino acid sequence selected from the group consisting of SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NOs:134-140, and combinations thereof.

Embodiment 68 is the method of any one of embodiments 65-67, wherein the cancer cell is contacted with:
(a) one, two, three, four, five, six, seven, eight, nine, or ten nucleic acids encoding an elephant LIF protein; or
(b) one, two, three, four, five, six, seven, eight, nine, or ten elephant LIF proteins.

Embodiment 69 is the method of any one of embodiments 65-68, wherein the one or more nucleic acids or the one or more proteins are in the form of a composition, wherein the composition further comprises a pharmaceutically acceptable carrier.

Embodiment 70 is the method of any one of embodiments 65-69, wherein the cancer cell is from a mammal.

Embodiment 71 is the method of embodiment 70, wherein the mammal is a human.

Embodiment 72 is the method of any one of embodiments 65-71, wherein the cancer cell is from a tumor or cancer selected from a lung cancer, a gastric cancer, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, and other solid tumors, and a non-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

### EXAMPLES

### Example 1: Identification of Elephant p53 and Elephant LIF ORFs

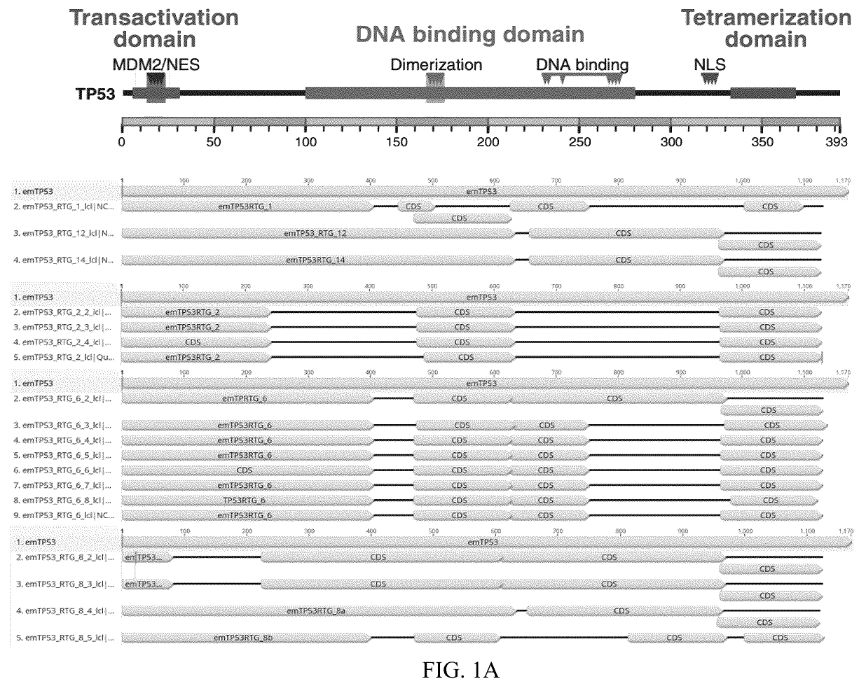

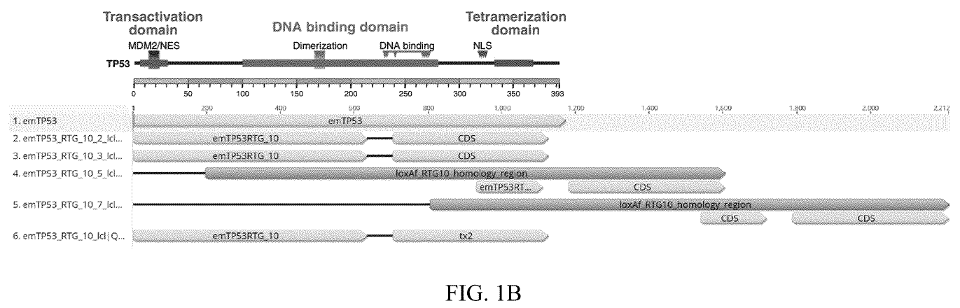

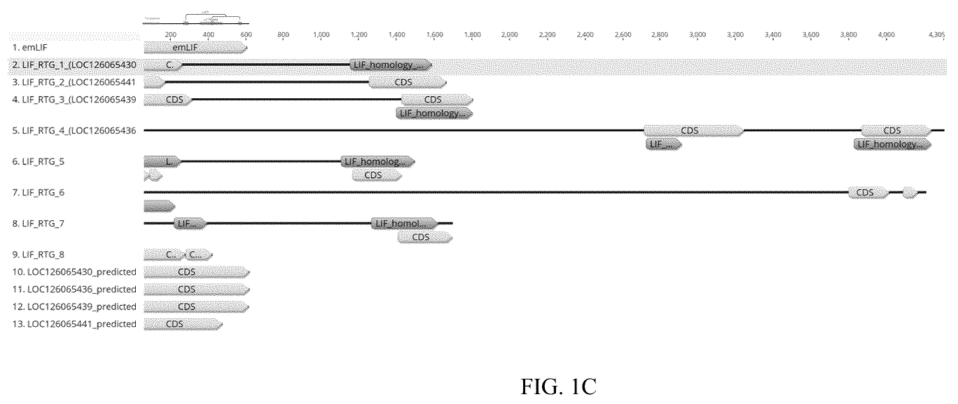

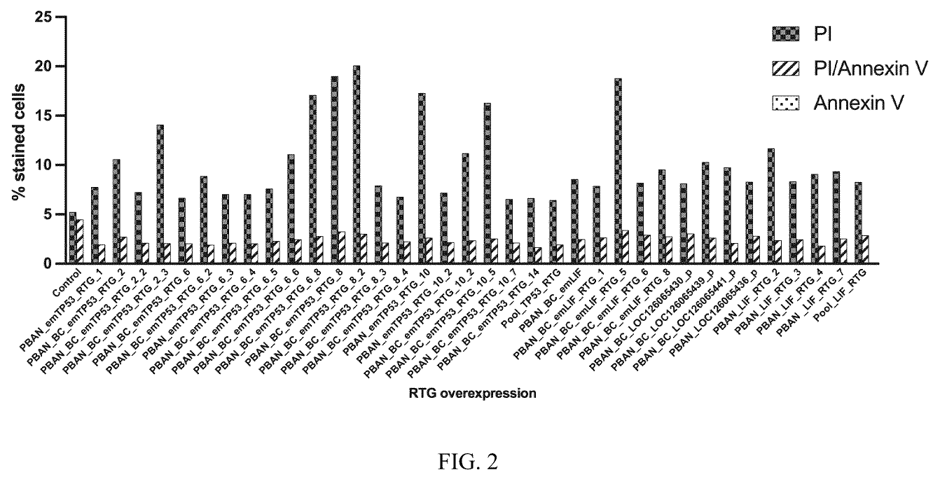

The elephant p53 and elephant LIF retrogene sequences were identified and translated to produce all possible open reading frames (ORFs) from each nucleotide sequence in Elephas maximus. Upon production of each possible ORF, an alignment was done using Blast-X (National Institutes of Health (NIH), Bethesda, MD) to the full length p53 retrogene sequence (FIGS. 1A and 1B) or the full length LIF retrogene sequence (FIG. 1C). Translated ORFs from the retrogenes that registered homology with full-length canonical sequences of p53 or LIF, respectively, were determined to be potentially expressed peptide relevant to the regulation of the full length sequences or other genetic networks in which they interact.

### Example 2: Creation of Expression Cassettes and Evaluating Transgene Expression

Elephas maximus TP53 and LIF retrogene sequences are cloned into barcoded, genome-integratable, overexpression cassette for testing. The overexpression cassette can be drug-inducible. The TP53 and LIF retrogene sequences are tested in combination. This allows for combinatorial screens and direct follow-up experiments testing a variety of different conditions with the same overexpression cassettes in many cell lines and culture/assay conditions. Over-expression of retrogene transcripts is validated by RNA sequencing where barcoded transcripts are identified.

### Example 3: Gain-of-Function Experiments on Human Primary Fibroblasts

Primary human cell lines are used and combinations of retrogene expression cassettes described in Example 2 are introduced into the primary human cell lines. Upon introduction of the retrogene expression cassettes, the primary human cell lines are subjected to standard assays for inducing cancer (SV40T, hTERT, irradiation), and cells that appear to have gained an ability to withstand cancer genotypes/phenotypes are sorted. These cells are sequenced to determine if the cells contain a retrogene expression cassette, and the identity of the retrogene is determined. This information is used to inform subsequent rounds of refined screens on specific combinations of retrogenes.

### Example 4: Validation Experiments on Human Cancer Cell Lines

Discovery of specific sets of important retrogenes able to produce a cancer-relevant phenotype are tested in human cancer cell lines. These cell lines are subjected to assays to validate that cells obtain enhanced apoptosis under certain carcinogenic conditions when elephant retrogenes are overexpressed. Specifically, retrogene over-expression combinations identified in Example 2 are introduced into human cancer cells lines (e.g., HeLa, HEK, K562). Cells are stained with apoptotic and pre-apoptotic cell markers, sorted, and sequenced with next-generation sequencing technologies (e.g., RNA-seq, ATAC-seq, single-cell sequencing). Identified combinations of retrogenes inducing apoptotic and pre-apoptotic markers are over-expressed in cancer cell lines to validate induced apoptosis as a function of elephant retrogene expression. These cancer cell lines are also re-screened with the broader set of retrogenes to identify retrogenes or sets of retrogenes that do not protect cells from becoming cancerous (as per Examples 2 and 3) but induce apoptosis in cell lines that already have cancerous genotype/phenotypes.

### Example 5: Loss-of-Function Experiments on Primary Elephant Cells

Based on the results from Examples 3 and 4, shRNAs that target specific retrogenes in elephant cells are generated, and the shRNAs are validated in elephant cells by knocking down the specific retrogene and performing cancer assays on the cells. shRNAs are cloned into transient expression plasmids and inducible, genome-integratable cassettes for expression in cells. The shRNA experiments are also combined with overexpression screens to follow up on results in Examples 2 and 3. Upon introduction of the retrogene expression cassettes, the primary human cell lines are subjected to standard assays for inducing cancer (SV40T, hTERT, irradiation), and cells that appear to have lost the ability to withstand cancer genotypes/phenotypes are sorted (i.e., cancer is created at an increased rate). Furthermore, standard pluripotency reprogramming assays (both chemical reprogramming and transgene transcription factor over-expression methods) with these transgene tools to demonstrate that these genes are a road-block for cellular-reprogramming and are utilized by cells to protect the germ-line. Reprogramming is validated via standard assay including NGS-validated expression of pluripotency factors, embryoid body formation, and differentiation into each germ-layer (endoderm, mesoderm, and ectoderm). The rationale for these experiments is that cancer and pluripotency have overlapping phenotypes and are both impacted by tumor suppressor retrogene expansion systems. Results from these experiments are also translated back to human cell systems via combinatorial elephant retrogene over-expansion to demonstrate improved cancer resistance, apoptosis under stress conditions, and enhanced germ-line protection.

### Example 6: Expression of Elephas maximus TP53 and/or LIF Retrogenes Resulted in Apoptosis and Necrosis of HEK293 Cells

Human cancer cells (HEK293-T) were transfected with selectable, genome-integrating, DOX-inducible overexpression cassettes containing Elephas maximus TP53 and/or LIF retrogenes. The HEK293-T cells were transfected with Elephas maximus LIF, LIF RTG_1 (SEQ ID NO:52), LIF RTG_2 (SEQ ID NO:54), LIF RTG_3 (SEQ ID NO:56), LIF RTG_4 (SEQ ID NO:58), LIF RTG_5 (SEQ ID NO:60), LIF RTG_6 (SEQ ID NO:62), LIF RTG_7 (SEQ ID NO:64), LIF RTG_8 (SEQ ID NO:66), LIF RTG_LOC126065436 (SEQ ID NO:70), LIF RTG_LOC126065439 (SEQ ID NO:72), LIF RTG_LOC126065441 (SEQ ID NO:74), LIF RTG_POOL, TP53 RTG_1 (SEQ ID NO:50), TP53 RTG_10 (SEQ ID NO:2), TP53 RTG_10.2 (SEQ ID NO:30), TP53 RTG_10.5 (SEQ ID NO:44), TP53 RTG_10.7 (SEQ ID NO:46), TP53 RTG_14 (SEQ ID NO:36), TP53 RTG_2 (SEQ ID NO:6), TP53 RTG_2.2 (SEQ ID NO:28), TP53 RTG_2.3 (SEQ ID NO:34), TP53 RTG_6 (SEQ ID NO:14), TP53 RTG_6.2 (SEQ ID NO:10), TP53 RTG_6.3 (SEQ ID NO:12), TP53 RTG_6.4 (SEQ ID NO:16), TP53 RTG_6.5 (SEQ ID NO:18), TP53 RTG_6.6 (SEQ ID NO:20), TP53 RTG_6.8 (SEQ ID NO:40), TP53 RTG_8 (SEQ ID NO:8), TP53 RTG_8.2 (SEQ ID NO:24), TP53 RTG_8.3 (SEQ ID NO:26), TP53 RTG_8.4 (SEQ ID NO:42), TP53 RTG_Pool, and TP53 RTG_Pool and LIF RTG_Pool, These cell lines were created using the Lonza nucleofection system (Lonza; Basel, Switzerland) and selected with Puromycin 3 days after nucleofection. Cell lines were recovered and passaged into new cell culture wells for testing. Cell lines were treated for 4 days with DOX induction, and then all cells (floating and attached) were collected and stained with the Dead Cell Apoptosis Kit with Annexin V FITC & Propidium Iodide (Invitrogen V13242; Invitrogen; Waltham, MA) to measure the percentage of stained cells as an indication of cell viability. PI+, PI+/Annexin V+ and Annexin V+ cells all indicate cells at various stages of apoptosis and necrosis. It was demonstrated that all TP53 and/or LIF retrogenes increased the PI positive population compared to non-transfected but double stained HEK293-T cells demonstrating that TP53 and/or LIF expression resulted in increased indicators of apoptosis and necrosis of HEK293-T cells.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

1. A method of treating or preventing cancer in a subject in need thereof, the method comprising administering to the subject in need thereof:
(a) one or more nucleic acids encoding an elephant p53 protein, wherein the one or more nucleic acids encoding the elephant p53 protein comprise a nucleotide sequence having at least 80% identity with a nucleotide sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, and combinations thereof; or
(b) one or more elephant p53 proteins, wherein the one or more elephant p53 proteins comprise an amino acid sequence with at least 85% identity to an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NOs:75-133, and combinations thereof.

2. The method of claim 1, wherein;
(a) the one or more nucleic acids encoding the elephant p53 protein comprise a nucleotide sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, and combinations thereof; or
(b) the one or more elephant p53 proteins comprise an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NOs:75-133, and combinations thereof.

3. (canceled)

4. The method of claim 1, wherein the subject in need thereof is administered
(a) one, two, three, four, five, six, seven, eight, nine, or ten nucleic acids encoding an elephant p53 protein; or
(b) one, two, three, four, five six, seven, eight, nine, or ten elephant p53 proteins.

5. The method of claim 1, wherein the subject in need thereof is further administered:
(c) one or more nucleic acids encoding an elephant leukemia inhibitory factor (LIF) protein, wherein the one or more nucleic acids encoding the elephant LIF protein comprise a nucleotide sequence having at least 80% identity with a nucleotide sequence selected from the group consisting of SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, and combinations thereof; or
(d) one or more elephant LIF proteins, wherein the one or more elephant LIF proteins comprise an amino acid sequence with at least 85% identity to an amino acid sequence selected from the group consisting of SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NOs:134-140, and combinations thereof.

6. The method of claim 5, wherein:
(a) the one or more nucleic acids encoding the elephant LIF protein comprise a nucleotide sequence selected from the group consisting of SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, and combinations thereof; or
(b) the one or more elephant LIF proteins comprise an amino acid sequence selected from the group consisting of SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NOs:134-140, and combinations thereof.

7. (canceled)

8. The method of claim 5, wherein the subject in need thereof is administered;
(c) one, two, three, four, five, six, seven, eight, nine, or ten nucleic acids encoding an elephant LIF protein; or
(d) one, two, three, four, five, six, seven, eight, nine, or ten elephant LIF proteins.

9. The method of claim 1, wherein the one or more nucleic acids or the one or more proteins are in the form of a composition, wherein the composition further comprises a pharmaceutically acceptable carrier.

10. The method of claim 1, wherein the subject in need thereof is a mammal.

11. The method of claim 10, wherein the mammal is a human.

12. The method of claim 1, wherein the cancer is selected from a lung cancer, a gastric cancer, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, and other solid tumors, and a non-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

13. A pharmaceutical composition comprising a pharmaceutically acceptable carrier and
(a) one or more nucleic acids encoding an elephant p53 protein, wherein the one or more nucleic acids encoding the elephant p53 protein comprise a nucleotide sequence having at least 80% identity with a nucleotide sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, and combinations thereof; or
(b) one or more elephant p53 proteins, wherein the one or more elephant p53 proteins comprise an amino acid sequence with at least 85% identity to an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NOs:75-133, and combinations thereof.

14. The pharmaceutical composition of claim 13, wherein;
(a) the one or more nucleic acids encoding the elephant p53 protein comprise a nucleotide sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, and combinations thereof; or
(b) the one or more elephant p53 proteins comprise an amino acid sequence selected from the group consisting of SEQ ID NO:2, SEQ ID NO:4, SEQ ID NO:6, SEQ ID NO:8. SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NOs:75-133, and combinations thereof.

15. (canceled)

16. The pharmaceutical composition of claim 13, wherein the composition comprises
(a) one, two, three, four, five, six, seven, eight, nine, or ten nucleic acids encoding an elephant p53 protein; or
(b) one, two, three, four, five six, seven, eight, nine, or ten elephant p53 proteins.

17. The pharmaceutical composition of claim 13, wherein the pharmaceutical composition further comprises:
(c) one or more nucleic acids encoding an elephant leukemia inhibitory factor (LIF) protein, wherein the one or more nucleic acids encoding the elephant LIF protein comprise a nucleotide sequence having at least 80% identity with a nucleotide sequence selected from the group consisting of SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, and combinations thereof; or
(d) one or more elephant LIF proteins, wherein the one or more elephant LIF proteins comprise an amino acid sequence with at least 85% identity to an amino acid sequence selected from the group consisting of SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NOs:134-140, and combinations thereof.

18. The pharmaceutical composition of claim 17, wherein;
(a) the one or more nucleic acids encoding the elephant LIF protein comprise a nucleotide sequence selected from the group consisting of SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, and combinations thereof; or
(b) the one or more elephant LIF proteins comprise an amino acid sequence selected from the group consisting of SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NOs:134-140, and combinations thereof.

19. (canceled)

20. The pharmaceutical composition of claim 17, wherein the pharmaceutical composition comprises:
(c) one, two, three, four, five, six, seven, eight, nine, or ten nucleic acids encoding an elephant LIF protein; or
(d) one, two, three, four, five, six, seven, eight, nine, or ten elephant LIF proteins.

21. A kit for treating or preventing cancer in a subject in need thereof, the kit comprising:
(a) one or more nucleic acids encoding an elephant p53 protein, wherein the one or more nucleic acids encoding the elephant p53 protein comprise a nucleotide sequence having at least 80% identity with a nucleotide sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, and combinations thereof; or
(b) one or more elephant p53 proteins, wherein the one or more elephant p53 proteins comprise an amino acid sequence with at least 85% identity to an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NOs:75-133, and combinations thereof; and
(c) instructions for use.

22-23. (canceled)

24. The kit of claim 21, wherein the kit comprises:
(a) one, two, three, four, five, six, seven, eight, nine, or ten nucleic acids encoding an elephant p53 protein; or
(b) one, two, three, four, five six, seven, eight, nine, or ten elephant p53 proteins.

25. The kit of claim 21, wherein the kit further comprises:
(d) one or more nucleic acids encoding an elephant leukemia inhibitory factor (LIF) protein, wherein the one or more nucleic acids encoding the elephant LIF protein comprise a nucleotide sequence having at least 80% identity with a nucleotide sequence selected from the group consisting of SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, and combinations thereof; or
(e) one or more elephant LIF proteins, wherein the one or more elephant LIF proteins comprise an amino acid sequence with at least 85% identity to an amino acid sequence selected from the group consisting of SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NOs:134-140, and combinations thereof.

26-27. (canceled)

28. The kit of claim 25, wherein the kit comprises:
(d) one, two, three, four, five, six, seven, eight, nine, or ten nucleic acids encoding an elephant LIF protein; or
(e) one, two, three, four, five, six, seven, eight, nine, or ten elephant LIF proteins.

29-32. (canceled)

33. A method of treating or preventing cancer in a subject in need thereof, the method comprising administering to the subject in need thereof:
(a) one or more nucleic acids encoding an elephant leukemia inhibitory factor (LIF) protein, wherein the one or more nucleic acids encoding the elephant LIF protein comprise a nucleotide sequence having at least 80% identity with a nucleotide sequence selected from the group consisting of SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, and combinations thereof; or
(b) one or more elephant LIF proteins, wherein the one or more elephant LIF proteins comprise an amino acid sequence with at least 85% identity to an amino acid sequence selected from the group consisting of SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NOs:134-140, and combinations thereof.

34. The method of claim 33, wherein;
(a) the one or more nucleic acids encoding the elephant LIF protein comprise a nucleotide sequence selected from the group consisting of SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, and combinations thereof, or
(b) the one or more elephant LIF proteins comprise an amino acid sequence selected from the group consisting of SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NOs:134-140, and combinations thereof.

35. (canceled)

36. The method of claim 33, wherein the subject in need thereof is administered:
(a) one, two, three, four, five, six, seven, eight, nine, or ten nucleic acids encoding an elephant LIF protein; or
(b) one, two, three, four, five, six, seven, eight, nine, or ten elephant LIF proteins.

37. The method of claim 33, wherein the one or more nucleic acids or the one or more proteins are in the form of a composition, wherein the composition further comprises a pharmaceutically acceptable carrier.

38. The method of claim 33, wherein the subject in need thereof is a mammal.

39. The method of claim 38, wherein the mammal is a human.

40. The method of claim 33, wherein the cancer is selected from a lung cancer, a gastric cancer, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, and other solid tumors, and a non-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

41. A pharmaceutical composition comprising a pharmaceutically acceptable carrier and
(a) one or more nucleic acids encoding an elephant leukemia inhibitory factor (LIF) protein, wherein the one or more nucleic acids encoding the elephant LIF protein comprise a nucleotide sequence having at least 80% identity with a nucleotide sequence selected from the group consisting of SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, and combinations thereof; or
(b) one or more elephant LIF proteins, wherein the one or more elephant LIF proteins comprise an amino acid sequence with at least 85% identity to an amino acid sequence selected from the group consisting of SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NOs:134-140, and combinations thereof.

42-44. (canceled)

45. A kit for treating or preventing cancer in a subject in need thereof, the kit comprising:
(a) one or more nucleic acids encoding an elephant leukemia inhibitory factor (LIF) protein, wherein the one or more nucleic acids encoding the elephant LIF protein comprise a nucleotide sequence having at least 80% identity with a nucleotide sequence selected from the group consisting of SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, and combinations thereof; or
(b) one or more elephant LIF proteins, wherein the one or more elephant LIF proteins comprise an amino acid sequence with at least 85% identity to an amino acid sequence selected from the group consisting of SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NOs:134-140, and combinations thereof; and
(c) instructions for use.

46-52. (canceled)


SEQUENCE LISTING (140 sequences)

SEQ ID NO: 1 - DNA, 1126 bp, synthetic construct
  source 1..1126: mol_type = other DNA
atggaggagcctcagtcagatctgagcattgagctccctctgagtcaagagacattttcatacttggggaaactcctttctgagaagctggttctatccccctcactgtccccagcagcggaggcagtagtcaatctgctactcccagaagatgctgcagactggctagaaagccaagctggggctcaaggaatatcagaagcccctacactagccacctcctggacgctgtcatcctctgtctcttctcagaagacctacccagcacctatcatttctgtctgggcttcttgcattctgggacagccaagtccgtcacctacacgtactcccctgaacttaacatgctgttttgccagctggcaaaggcctgtccagtgcagccatgggtcacctcaacacccccgcccagcacctgtgttcacaccatggccatctaccagacgtcagcatatgatggaggtcgtgaagcactgcccccaccttgagtgccgctctgactatagcgattgcttggaccctcctcagcacctcatgcagtgggaggaaacctgcatgctgagtatttggaggacaccatcactctatgacatagtgtggggtgccctaggagccaccagaggtcggttctgactaccaccatccacttcaacttcatgtgtaacagctcctgcatggggcgcatgaacccattctcaccattatgacaatggaagactccaatggtaatccgctgggacacaacagtttcaaggtgcatatttgtacctgtcctgggagacacagatgtacagaggaagacaatttccgcaagaagtgggagccttgccctgagccaccctctgggaggatcactaagcaaacactgcccaccagcaccagctcctctacgaagccaaagaagaagccactggatgaaaaatacttcacccttcagatccatgggcatgaatgtttcaagatgttcctaaagctcaatgaggccttggagctgaaggatgcccaggctgggaagcagccagaggggagcagggctcaatccagccttcccaagtctaagaaaaggcaatctacctcccgccataaaaaactaatgttcaagagagagcagcctgactcagactga

SEQ ID NO: 2 - protein, 210 aa, synthetic construct
MEEPQSDLSIELPLSQETFSYLGKLLSEKLVLSPSLSPAAEAVVNLLLPEDAADWLESQAGAQGISEAPTLATSWTLSSSVSSQKTYPAPIISVWASCILGQPSPSPTRTPLNLTCCFASWQRPVQCSHGSPQHPRPAPVFTPWPSTRRQHMMEVVKHCPHLECRSDYSDCLDPPQHLMQWEETCMLSIWRTPSLYDIVWGALGATRGRF

SEQ ID NO: 3 - DNA, 1126 bp, synthetic construct
  source 1..1126: mol_type = other DNA
atggaggagcctcagtcagatctcagcactgagctccctctgagtcaagagacgttttcatacttggggaaactccttcctgagaagctggttctgtccccctcactgtccccagcagcagaggcagtagacgatctgctactcccagaagatgctgcagactggctagaaagccaagctggggctcaaggaatatcagaagcccctacactagccacctcctggacgctgtcatcctctgtcccttctcagaagacctacccagcacctatcgtttctgtctgggcttcttgcattctgggacagccaagtccgtcacctacacgtactcccctgaacttaacatgctgttttgccagctggcaaaggcctgtccagtgcagccgtgggtcacctcaacacccccgcccagcacctgtgttcacaccatggccatctaccagacatcagcatatgatggaggtcgtgaagcactgccgccaccttgagtgccactctgactatagcgattgcttggaccctcctcagcacctcatgcagtgggaggaaacctgcatgctgagtatttggaggacaccatcactctatgacatagtgtggggtgccctaggagccaccagaggtcggttctgactaccaccatccacttcaacttcatgtgtaacagctcctgcatggggagcatgaacccatcctcaccatcatcacactggaagactccaatggtaatccgctgggacacaacagtttcgaggtgcatatttgtacctgtcctgggagacacagatgtacagaggaagacaatttccacaagaagtgggagccttgccctgagccaccctctgggaggatcactaagcaaactctgcccaccagcaccagctcctctaccaagccaaagaagaagccactggatgaaaaatacttcacccttcagatccatgggcatgaatgcttcaagatgttcctaaagctcaacgaggccttggagctgaaggatgcccaggctgggaagcagccagaggggagcagggctcaatccagccttcccaagtctaagaaaaggcaatctacctcctgccataaaaaactaatgttcaagagagagcagcctgactcagactga

SEQ ID NO: 4 - protein, 210 aa, synthetic construct
MEEPQSDLSTELPLSQETFSYLGKLLPEKLVLSPSLSPAAEAVDDLLLPEDAADWLESQAGAQGISEAPTLATSWTLSSSVPSQKTYPAPIVSVWASCILGQPSPSPTRTPLNLTCCFASWQRPVQCSRGSPQHPRPAPVFTPWPSTRHQHMMEVVKHCRHLECHSDYSDCLDPPQHLMQWEETCMLSIWRTPSLYDIVWGALGATRGRF

SEQ ID NO: 5 - DNA, 1128 bp, synthetic construct
  source 1..1128: mol_type = other DNA
atggaggagcctcagtcagatctcagtactgagctctttctgagtcaagagaccttttcgtacttgggaaaactccttcctgagaagctggttctgtccccctcactgtccccagcagcggaggcagtagacgatctgctactcccagaagatgctgcagactggctagaaagccaagctggggctcaaggaatatcagcagcccctgcaccagccacccttacaccagccacctcctagacactttcatcctctgtcccttcccagaagacctactgcagcaactgtggtttccatcttggcttcctgcattctgggacagccaagtctgtcacctgcacgtactcccctgaccttaacaagctgttttgccagctggcaaagacctgtccagtgcagccgtagctcagctcaccaccccaccccaccccagcacctgtgttcacaccatggccatctaccagacgtcagcatacgacagaggtcatgcagcactgcccccaccttgagtgctactccgactatagtgatggcctggccgctcctcagcatcttatccaggtgggaggaatcctgcgtgctgatatttgtaggacaccattactcttcgacatagtgtgggttaccctatgagctacctcaggtcggttctgactaccaccatccacttcatgtgtagcagctcctgcatggggggggggaacccatcctcaccatcatcacactggaagactccgatggtaatctgctaggacacaacagtttcgaggtgcatatttgtactgttctgggagagacagatgtacagaggaagaaaatttccacaacaagtgggagccaccctctgagaggatcactaagtaagcactgcccaccagcaccagctcctctaccgagccaaagaagaagccagtggatgaaaaatatttcacccttaagatccgtgggcatgaatgcttcaagatgttcctagagttgaacgaggcattggagctgaaggatgcccaggctgggaagcagccagaggggagcagggctcaatgcagccttccaaactctaagaaaggggaatctaccacccactgtaaaaaactaatgttcaagagagaggggcctgactcagactga

SEQ ID NO: 6 - protein, 79 aa, synthetic construct
MEEPQSDLSTELFLSQETFSYLGKLLPEKLVLSPSLSPAAEAVDDLLLPEDAADWLESQAGAQGISAAPAPATLTPATS

SEQ ID NO: 7 - DNA, 1124 bp, synthetic construct
  source 1..1124: mol_type = other DNA
atggaggagcctcagtcagatctcagcactgagctccctctgagtcgagacgttttcatacttggggaaactccttcctgagaagctggttctgtccccctcactgtccccagcagcagaggcagtagacgatctgctactcccagaagatgctgcagactggctagaaagccaagctggggctcaaggaatatcagaagcgcctacactagccacctcctggatgctgtcatcctctgtcccttctcagaagacctacccagcacctatcgtttctgtctgggcttcttgcattctgggacagccaagtccgtcacctacacgtactcccctgaacttaacatgctgttttgccagctggcaaaggcctgtccagtgcagccgtgggtcacctcaacacccccgcctagcacctgtgttcacaccatggccatctatcagacgtcagcatatgatggaggtcgtgaagcactgccgccagcttgagtgccactctgactatagcgattgcttggaccctcctcagcacctcatgcagtgggaggaaacctgcatgctgagtatttggaggacaccatcactctatgacacagtgtggggtgccctatgagccaccagaggtcggttctgactaccaccattcacttcaacttcatgtgtaacagctcctgcatggggagcatgaacccatcctcaccatcatcacactggaagactccaatggtaatccgctgggacacaacacttttgaggtgcatatttgtacctgtcccgggagacacagatgtacagaggaagacaatttccacaagaagtgggagccttgccctgagccaccctctgggaggatcactaagcaaacactgcccaccagcaccagctcctctaccaagccaaagaagaagccactggatgaaaaatacttcacccttcagatccatgggcatgaatgcttcaagatgttcctaaagctcaacgaggccttggagctgaaggatgcccaggctgggaagcagccagaggggagcagggctcaatccagccttcccaagtctaagaaaaggcaatctacctcccgccataaaaaactaacattcaagagagagcagcctgactcagactga

SEQ ID NO: 8 - protein, 26 aa, synthetic construct
MEEPQSDLSTELPLSRDVFILGETPS

SEQ ID NO: 9 - DNA, 1130 bp, synthetic construct
  source 1..1130: mol_type = other DNA
atggaggagcccaagtcagatctcagtactgagctccctctgagtcaagagactttttcatacttggggaaactccttcctgagaagctggttctgtccccctcactgtccccagcagcggaggcagtagacgatctgctgctcccaggagatgctgcagactgcctagaaagccaagctggggctcaaggaatatcagcagcccctgcaccagcctcacttacaccagccacctcctggacactctcatcctctgtcccttcccagaagacctactgcagcaactgtggtttccgtcttggcttcctgcattctgggacatccaagtctgtcacctgcatgtactcccctggccttaacaagctgttttgccagctggcaaagacctgtccaatgcagccgtagctcagctcaccaccccaccccagcacctgtgttcacatcatggccatctaccagacgtcagcatatgacagaggtcgtgcagcactgcccccaccttgagtgctgctccgactatagcgatggcctggccgctcctcagcatcttatccaggtgggaggaatcctgcgtgctgatatttgtaggacaccatcactcttcgacatagtgtggggtaccctatgagctacctcaggtcggttctgactaccaccatccacttcaacttcatgtgtagcagctccagcatggcgggtggggaacccatcctcaccatcatcacactggaagactccgatggtaatctgctaggacacaacagtttcgaggtgcatatttgtactgttctgggagagacagacgtacagaggaagaaaatttccacaacaagtgggagccaccctctgagaggatcactaagtaagcactgcacaccagcaccagctcctctaccgagccaaagaagaagccactggatgaaaaatatttcacccttaagatccgtgggcatgaatgcttcaagatgttcctagagttgaatgaggcattggagctgaaggatgcccaggctaggaagcagccagaggggagcagggctcaatgcagccttccaaactctaagaaaggggaatctaccacccactgtaaaaaactaatgttcaagagagaggggcctgactcagactga

SEQ ID NO: 10 - protein, 134 aa, synthetic construct
MEEPKSDLSTELPLSQETFSYLGKLLPEKLVLSPSLSPAAEAVDDLLLPGDAADCLESQAGAQGISAAPAPASLTPATSWTLSSSVPSQKTYCSNCGFRLGFLHSGTSKSVTCMYSPGLNKLFCQLAKTCPMQP

SEQ ID NO: 11 - DNA, 1136 bp, synthetic construct
  source 1..1136: mol_type = other DNA
atggagaagcccgagtcagatctcagtactgagctccctctgaatcaagagactttttcgtacttggggaaactccttcctgagaagctggttctgtccccctcactgtcccccgcagcggaggcagtagacgatctgctactcccagaagatgctgcagactggctagaaagccaagctggggctcaaggaatatcagcagcccctgcaccagccacccttacaccagccacctcctggacactctcatcctctgtcccttcccagaagacctactgcagcaactgtggtttccgtcttggcttcctgcattctgggacagccaagtctgtcacctgcatgtactcccctggccataacaaactgttttgccagctggcaaagacctgtccagtgcagtcgtagctcagctcaccaccccaccccaccccagcacctgtgttcacaccatggccatctaccagatgtcagcatatgacagaggtcgtgcagcactgcccccaccttgagtgctgctccgactatagcgatggcctggccgctcctcagcatcttatccaggtgggaggaatcctgcatgctgatatttgtaggacaccatcactcttcgacatagtgtggggtaccctatgagctacctcaggtcggttctgactaccaccatccacttcaacttcatgtgtagcagctcctgcatggggggggggggaacccatcctcaccatcatcacactggaagactccgatggtaatctgctaggacacaacagtttcgaggtgcatatttgtactgttccgggagagacagtcgtacagaggaagaaaatttccacaacaagtgggagccaccctctgagaggatcactaagtaagcactgcacaccagcaccagctcctctaccaagccaaagaagaagccagtggatgaaaaatatttcacccttaagatccgtgggcatgaatgcttcaagatgttcctagagttgaatgaggcattggagctgaaggatgcccaggctgggaatcagccagagagcagcagggatcaatgcagccttccaaactctaagaaaggggaatctaccacccactgtaaaaaactaacgttcaagagagaggggcctgactcagactga

SEQ ID NO: 12 - protein, 134 aa, synthetic construct
MEKPESDLSTELPLNQETFSYLGKLLPEKLVLSPSLSPAAEAVDDLLLPEDAADWLESQAGAQGISAAPAPATLTPATSWTLSSSVPSQKTYCSNCGFRLGFLHSGTAKSVTCMYSPGHNKLFCQLAKTCPVQS

SEQ ID NO: 13 - DNA, 1129 bp, synthetic construct
  source 1..1129: mol_type = other DNA
atggaggagcccaagtcagatctcagtactgagctccctctgagtcaagagactttttcatacttggggaaactccttcctgagaagctggttctgtccctctcactgtccccagcagcggaggcagtagacgatctgctgctcccaggagatgctgcagactgcctagaaagccaagctggggctcaaggaatatcagcagcccctgcaccagccacacttacaccagccacctcctggacactctcatcctctgtcccttcccagaagacctactgcagcaactgtggtttccgtcttggcttcctgcattctgggacatccaagtctgtcacctgcatgtactcccctggccttaacaagctgttttgccagctggcaaagacctgtccagtgcagccgtagctcagctcaccaccccaccccagcacctgtgttcacaccatggccatctaccagacgtcagcatatgacagaggtcgtgcagcactgcccccaccttgagtgctgctccgactatagcgatggcctggccgctcctcagcatcttatccaggtgggaggaatcctgcgtgctgatatttgtaggacaccatcactcttcgacatagtgtggggtaccctatgagctacctcaggtcggttctgactaccaccatccacttcaacttcatgtgtagcagctcctgcgtgggggcgggaaacccatcctcaccatcatcacactggaagactccgatggtaatctgctaggacacaacagtttcgaggtgcatatttgtactgttctgggagagacagacgtacagaggaagaaaatttccacaacaagtgggagccaccctctgagaggatcactaagtaagcactgcacaccagcaccagctcctctaccgagccaaagaagaagccagtggatgaaaaatatttcacccttaagatccgtgggcatgaatgcttcaagatgttcctagagttgaatgaggcattggagctgaaggatgcccaggctgggaagcagccagaggggagcagggctcagtgcagccttccaaactctaagaaaagggaatctaccacccactgtaaaaaactaatgttcaagagagaggggcctgactcagactga

SEQ ID NO: 14 - protein, 134 aa, synthetic construct
MEEPKSDLSTELPLSQETFSYLGKLLPEKLVLSLSLSPAAEAVDDLLLPGDAADCLESQAGAQGISAAPAPATLTPATSWTLSSSVPSQKTYCSNCGFRLGFLHSGTSKSVTCMYSPGLNKLFCQLAKTCPVQP

SEQ ID NO: 15 - DNA, 1129 bp, synthetic construct
  source 1..1129: mol_type = other DNA
atggaggagcccaagtcagatctcagtactgagctccctctgagtcaagagactttttcatacttggggaaactccttcctgagaagctggttctgtccctctcactgtccccagcagcggaggcagtagacgatctgctgctcccaggagatgctgcagactgcctagaaagccaagctggggctcaaggaatatcagcagcccctgcaccagccacacttacaccagccacctcctggacactctcatcctctgtcccttcccagaagacctactgcagcaactgtggtttccgtcttggcttcctgcattctgggacatccaagtctgtcacctgcatgtactcccctggccttaacaagctgttttgccagctggcaaagacctgtccagtgcagccgtagctcagctcaccaccccaccccagcacctgtgttcacaccatggccatctaccagacgtcagcatatgacagaggtcgtgcagcactgcccccaccttgagtgctgctccgactatagcgatggcctggccgctcctcagcatcttatccaggtgggaggaatcctgcgtgctgatatttgtaggacaccatcactcttcgacatagtgtggggtaccctatgagctacctcaggtcggttctgactaccaccatccacttcaacttcatgtgtagcagctcctgcgtgggggcgggaaacccatcctcaccatcatcacactggaagactccaatggtaatctgctaggacacaacagtttcgaggtgcatatttgtactgttctgggagagacagacgtacagaggaagaaaatttccacaacaagtgggagccaccctctgagaggatcactaagtaagcactgcacaccagcaccagctcctctaccgagccaaagaagaagccagtggatgaaaaatatttcacccttaagatccgtgggcatgaatgcttcaagatgttcctagagttgaatgaggcattggagctgaaggatgcccaggctgggaagcagccagaggggagcagggctcaatgcagccttccaaactctaagaaaagggaatctaccacccactgtaaaaaactaatgttcaagagagaggggcctgactcagactga

SEQ ID NO: 16 - protein, 134 aa, synthetic construct; identical to SEQ ID NO: 14

SEQ ID NO: 17 - DNA, 1129 bp, synthetic construct
  source 1..1129: mol_type = other DNA
atggaggagcccaagtcagatctcagtactgagctccctctgagtcaagagactttttcatacttggggaaactccttcctgagaagctggttctgtccccctcactgtccccagcagcggaggcagtagactatctgctgctcccaggagatgctgcagactgcctagaaagccaagctggggctcaaggaatatcagcagcccctgcaccagccacccttacaccagccacctcctggacactctcatcctctgtcccttcccagaagacctactgcagcaactgtggtttccgtcttggcttcctgcattctgggacagccaagtctgtcacctgcatgtactcccctggccttaacaagctgttttgccagctggcaaagacctgtccagtgcagccgtagctcagctcaccaccccaccccagcacctgtgttcacaccatggccatctaccagacgtcagcatatgacagaggtcgtgcagcactgcccccaccttgagtgctgctccgactatagcgatggcctggccgctcctcagcatcttatccaagtgggaggaatcctgcgtgctgatatttgtaggacaccatcactcttcgacatagtgtggggtaccctatgagctacctcaggtcggttctgactaccaccatccacttcaacttcatgtgtagcagctcctgcatggcgggggggaacccatcctcaccatcatcacactggaagactccgatggtaatctgctaggacacaacagtttcgaggtgcatatttgtactgttctgggagagacagacgtacagaggaagaaaatttccacaacaagtgggagccaccctctcagaggatcactaagtaagcactgcacaccagcaccagctcctctaccgagccaaagaagaagccagtggatgaaaaatatttcacccttaagatccgtgggcatgaatgcgtcaagatgttcctagagttgaatgaggcattggagctgaaggatgcccaggctgggaagcagccagaggggagcagggctcaatgcagccttccaaactctaagaaaggggaatctaccacccactgtaaaaaacgaatgttcaagagagaggggcctgactcagactga

SEQ ID NO: 18 - protein, 134 aa, synthetic construct
MEEPKSDLSTELPLSQETFSYLGKLLPEKLVLSPSLSPAAEAVDYLLLPGDAADCLESQAGAQGISAAPAPATLTPATSWTLSSSVPSQKTYCSNCGFRLGFLHSGTAKSVTCMYSPGLNKLFCQLAKTCPVQP

SEQ ID NO: 19 - DNA, 1129 bp, synthetic construct
  source 1..1129: mol_type = other DNA
atggaggagcccaagtcagatctcagtactgagctccctctgagtcaagagactttttcatacttggggaaactccttcctgagaagctggttctgtccctctcactgtccccagcagcggaggcagtagacgatctgctgctcccaggagatgctgcagactgcctagaaagccaagctggggctcaaggaatatcagcagcccctgcaccagccacacttacaccagccacctcctggacactctcatcctctgtcccttcccagaagacctactgcagcaactgtggtttccgtcttggcttcctgcattctgggacatccaagtctgtcacctgcatgtactcccctggccttaacaagctgttttgccagctggcaaagacctgtccagtgcagccgtagctcagctcaccaccccaccccagcacctgtgttcacaccatggccatctaccagacgtcagcatatgacagaggtcgtgcagcactgcccccaccttgagtgctgctccgactatagcgatggcctggccgctcctcagcatcttatccaggtgggaggaatcctgcgtgctgatatttgtaggacaccatcactcttcgacatagtgtggggtaccctatgagctacctcaggtcggttctgactaccaccatccacttcaacttcatgtgtagcagctcctgcgtgggggcgggaaacccatcctcaccatcatcacactggaagactccgatggtaatctgctaggacacaacagtttcgaggtgcatatttgtactgttctgggagagacagacgtacagaggaagaaaatttccacaacaagtgggagccaccctctgagaggatcactaagtaagcactgcacaccagcaccagctcctctaccaagccaaagaagaagccagtggatgaaaaatatttcacccttaagatccgtgggcatgaatgcttcaagatgttcctagagttgaatgaggcattggagctgaaggatgcccaggctgggaagcagccagaggggagcagggctcaatgcagccttccaaactctaagaaaagggaatctaccacccactgtaaaaaactaatgttcaagagagaggggcctgactcagactga

SEQ ID NO: 20 - protein, 134 aa, synthetic construct; identical to SEQ ID NO: 14

SEQ ID NO: 21 - DNA, 1129 bp, synthetic construct
  source 1..1129: mol_type = other DNA
atggaggagcccaagtcagatctcagtactgagctccctctgagtcaagagactttttcatacttggggaaactccttcctgagaagctggttctgtccccctcactgtccccagcagcggaggcagtagacgatctgctgctcccaggagatgctgcagactgcctagaaagccaagctggggctcaaggaatatcagcagcccctgcaccagctacccttacaccagccacctcctggacactctcatcctctgtcccttcccagaagacctactgcagcaactgtggtttccgtcttggcttcctgcattctgggacatccaagtctgtcacctgcatgtactcccctggccttaacaagctgttttgccagctggcaaagacctgtccagtgcagccgtagctcagctcaccaccctaccccagcacctgtgttcacaccatggccatctaccagacgtcagcatatgacagaggtcgtgcagcactgcccccaccttgagtgctgctccgactatagcgatggcctggccgctcctcagcatcttatccaagtgggaggaatcctgcgtgctgatatttgtaggacaccatcactcttcgacatagtgtggggtaccctatgagctacctcaggtcggttctgactaccaccatccacttcaacttcatgtgtagcagctcctgcgtgggggcgggaaacccatcctcaccatcatcatactggaaaactccgatggtaatctgctagggcacaacagtttcgaggtgcatatttgtactgttctgggagagacagacgtacagaggaagaaaatttccacaacaagtgggagccaccctctgagaggatcactaagtaagcactgcacaccagcaccagctcctctaccgagccaaagaagaagccagtggatgaaaaatatttcacccttaagatccgtgggcatgaatgcttcaagatgttcctagagttgaatgaggcattggagctgaaggatgcccaggctgggaagcagccagaggggagcagggctcaatgcagccttccaaactctaagaaaggggaatctaccacccactgtaaaaaactaatgttcaagagagaggggcctgactcagactga

SEQ ID NO: 22 - protein, 134 aa, synthetic construct
MEEPKSDLSTELPLSQETFSYLGKLLPEKLVLSPSLSPAAEAVDDLLLPGDAADCLESQAGAQGISAAPAPATLTPATSWTLSSSVPSQKTYCSNCGFRLGFLHSGTSKSVTCMYSPGLNKLFCQLAKTCPVQP

SEQ ID NO: 23 - DNA, 1124 bp, synthetic construct
  source 1..1124: mol_type = other DNA
atggaggagcctcagtcagatctcagcactgagctccctctgagtcgagacgttttcatacttggggaaactccttcctgagaagctggttctgtccccctcactgtccccagcagcagaggcagtagacgatctgctactcccagaagatgctgcagactggctagaaagccaagctggggctcaaggaatatcagaagcgcctacactagccacctcctggatgctgtcatcctctgtcccttctcagaagacctacccagcacctatcgtttctgtctgggcttcttgcattctgggacagccaagtccgtcacctacacgtactcccctgaacttaacatgctgttttgccagctggcaaaggcctgtccagtgcagccgtgggtcacctcaacacccccgcctagcacctgtgttcacaccatggccatctatcagacgtcagcatatgatggaggtcgtgaagcactgccgccaccttgagtgccactctgactatagcgattgcttggaccctcctcagcacctcatgcagtgggaggaaacctgcatgctgagtatttggaggacaccatcactctatgacacagtgtggggtgccctatgagccaccagaggtcggttctgactaccaccattcacttcaacttcatgtgtaacagctcctgcatggggagcatgaacccatcctcaccatcatcacactggaagactccaatggtaatccgctgggacacaacacttttgaggtgcatatttgtacctgtcccgggagacacagatgtacagaggaagacaatttccacaagaagtgggagccttgccctgagccaccctctgggaggatcactaagcaaacactgcccaccagcaccagctcctctaccaagccaaagaagaagccactggatgaaaaatacttcacccttcagatccatgggcatgaatgcttcaagatgttcctaaagctcaacgaggccttggagctgaaggatgcccaggctgggaagcagccagaggggagcagggctcaatccagccttcccaagtctaagaaaaggcaatctacctcccgccataaacaacttatgttcaagagagagcagcctgactcagactga

SEQ ID NO: 24 - protein, 26 aa, synthetic construct
MEEPQSDLSTELPLSRDVFILGETPS

SEQ ID NO: 25 - DNA, 1124 bp, synthetic construct
  source 1..1124: mol_type = other DNA
atggaggagcctcagtcagatctcagcactgagctccctctgagtcgagacgttttcatacttggggaaactccttcctgagaagctggttctgtccccctcactgtccccagcagcagaggcagtagacgatctgctactcccagaagatgctgcagactggctagaaagccaagctggggctcaaggaatatcagaagcgcctacactagccacctcctggatgctgtcatcctctgtcccttctcagaagacctacccagcaactatcgtttctgtctgggcttcttgcattctgggacagccaagtccgtcacctacacgtactcccctgaacttaacatgctgttttgccagctggcaaaggcctgtccagtgcagccgtgggtcacctcaacacccccgcctagcacctgtgttcacaccatggccatctatcagacgtcagcatatgatggaggtcgtgaagcactgccgccaccttgagtgccactctgactatagcgattgcttggaccctcctcagcacctcatgcagtgggaggaaacctgcatgctgagtatttggaggacaccatcactctatgacacagtgtggggtgccctatgagccaccagaggtcggttctgactaccaccattcacttcaacttcatgtgtaacagctcctgcatggggagcatgaacccatcctcaccatcatcacactggaagactccaatggtaatccgctgggacacaacacttttgaggtgcatatttgtacctgtcccgggagacacagatgtacagaggaagacaatttccacaagaagtgggagccttgccctgagccaccctctgggaggatcactaagcaaacactgcccaccagcaccagctcctctaccaagccaaagaagaagccactggatgaaaaatacttcacccttcagatccatgggcatgaatgcttcaagatgttcctaaagctcaacgaggccttggagctgaaggatgcccaggctgggaagcagccagaggggagcagggctcaatccagccttcccaagtctaagaaaaggcaatctacctcccgccataaaaaactaacattcaagagagagcagcctgactcagactga

SEQ ID NO: 26 - protein, 26 aa, synthetic construct
MEEPQSDLSTELPLSRDVFILGETPS

SEQ ID NO: 27 - DNA, 1127 bp, synthetic construct
  source 1..1127: mol_type = other DNA
atggaggagcctcagtcagatctcagtactgagctctttctgagtcaagagaccttttcgtacttgggaaaactccttcctgagaagctggttctgtccccctcactgtccccagcagcggaggcagtagacgatctgctactcccagaagatgctgcagactggctagaaagccaagctggggctcaaggaatatcagcagcccctgcaccagccacccttacaccagccacctcctagacactttcatcctctgtcccttcccagaagacctactgcagcaactgtggtttccatcttggcttcctgcattctgggacagccaagtctgtcacctgcacgtactcccctgaccttaacaagctgttttgccagctggcaaagacctgtccagtgcagccgtagctcagctcaccaccccaccccaccccagcacctgtgttcacaccatggccatctaccagacgtcagcatatgacagaggtcatgcagcactgcccccaccttgagtgctactccgactatagtgatggcctggccgctcctcagcatcttatccaggtgggaggaatcctgcgtgctgatatttgtaggacaccattactcttcgacatagtgtgggttaccctatgagctacctcaggtcggttctgactaccaccatccacttcatgtgtagcagctcctgcatgggggggggaacccatcctcaccatcatcacactggaagactccgatggtaatctgctaggacacaacagtttcgaggtgcatatttgtactgttctgggagagacagatgtacagaggaagaaaatttccacaacaagtgggagccaccctctgagaggatcactaagtaagcactgcccaccagcaccagctcctctaccgagccaaagaagaagccagtggatgaaaaatatttcacccttaagatccgtgggcatgaatgcttcaagatgttcctagagttgaacgaggcattggagctgaaggatgcccaggctgggaagcagccagaggggagcagggctcaatgcagccttccaaactctaagaaaggggaatctaccacccactgtaaaaaactaatgttcaagagagaggggcctgactcagactga

SEQ ID NO: 28 - protein, 79 aa, synthetic construct; identical to SEQ ID NO: 6

SEQ ID NO: 29 - DNA, 1126 bp, synthetic construct
  source 1..1126: mol_type = other DNA
atggaggagcctcagtcagatctgagcattgagctccctctgagtcaagagacattttcatacttggggaaactcctttctgagaagctggttctatccccctcactgtccccagcagcggaggcagtagtcaatctgctactcccagaagatgctgcagactggctagaaagccaagctggggctcaaggaatatcagaagcccctacactagccacctcctggacgctgtcatcctctgtctcttctcagaagacctacccagcacctatcatttctgtctgggcttcttgcattctgggacagccaagtccgtcacctacacgtactcccctgaacttaacatgctgttttgccagctggcaaaggcctgtccagtgcagccatgggtcacctcaacacccccgcccagcacctgtgttcacaccatggccatctaccagacgtcagcatatgatggaggtcgtgaagcactgcccccaccttgagtgccgctctgactatagcgattgcttggaccctcctcagcacctcatgcagtgggaggaaacctgcatgctgagtatttggaggacaccatcactctatgacatagtgtggggtgccctaggagccaccagaggtcggttctgactaccaccatccacttcaacttcatgtgtaacagctcctgcatggggcgcatgaacccattctcaccattatgacaatggaagactccaatggtaatccgctgggacacaacagtttcaaggtgcatatttgtacctgtcctgggagacacagatgtacagaggaagacaatttccataagaagtgggagccttgccctgagccaccctctgggaggatcactaagcaaacactgcccaccagcaccagctcctctacgaagccaaagaagaagccactggatgaaaaatacttcacccttcagatccatgggcatgaatgtttcaagatgttcctaaagctcaatgaggccttggagctgaaggatgcccaggctgggaagcagccagaggggagcagggctcaatccagccttcccaagtctaagaaaaggcaatctacctcccgccataaaaaactaatgttcaagagagagcagcctgactcagactga

SEQ ID NO: 30 - protein, 210 aa, synthetic construct; identical to SEQ ID NO: 2

SEQ ID NO: 31 - DNA, 1126 bp, synthetic construct
  source 1..1126: mol_type = other DNA
atggaggagcctcagtcagatctgagcattgagctccctctgagtcaagagacattttcatacttggggaaactcctttctgagaagctggttctatccccctcactgtccccagcagcggaggcagtagtcaatctgctactcccagaagatgctgcagactggctagaaagccaagctggggctcaaggaatatcagaagcccctacactagccacctcctggacgctgtcatcctctgtctcttctcagaagacctacccagcacctatcatttctgtctgggcttcttgcattctgggacagccaagtccgtcacctacacgtactcccctgaacttaacatgctgttttgccagctggcaaaggcctgtccagtgcagccatgggtcacctcaacacccccgcccagcacctgtgttcacaccatggccatctaccagacgtcagcatatgatggaggtcgtgaagcactgcccccaccttgagtgccactctgactatagcgattgcttggaccctcctcagcacctcatgcagtgggaggaaacctgcatgctgagtatttggaggacaccatcactctatgacatagtgtggggtgccctaggagccaccagaggtcggttctgactaccaccatccacttcaacttcatgtgtaacagctcctgcatggggcgcatgaacccattctcaccattatgacaatggaagactccaatggtaatccgctgggacacaacagtttcaaggtgcatatttgtacctgtcctgggagacacagatgtacagaggaagacaatttccgcaagaagtgggagccttgccctgagccaccctctgggaggatcactaagcaaacactgcccaccagcaccagctcctctacgaagccaaagaagaagccactggatgaaaaatacttcacccttcagatccatgggcatgaatgtttcaagatgttcctaaagctcaatgaggccttggagctgaaggatgcccaggctgggaagcagccagaggggagcagggctcaatccagccttcccaagtctaagaaaaggcaatctacctcccgccataaaaaactaatgttcaagagagagcagcctgactcagactga

SEQ ID NO: 32 - protein, 210 aa, synthetic construct
MEEPQSDLSIELPLSQETFSYLGKLLSEKLVLSPSLSPAAEAVVNLLLPEDAADWLESQAGAQGISEAPTLATSWTLSSSVSSQKTYPAPIISVWASCILGQPSPSPTRTPLNLTCCFASWQRPVQCSHGSPQHPRPAPVFTPWPSTRRQHMMEVVKHCPHLECHSDYSDCLDPPQHLMQWEETCMLSIWRTPSLYDIVWGALGATRGRF

SEQ ID NO: 33 - DNA, 1128 bp, synthetic construct
  source 1..1128: mol_type = other DNA
atggaggagcctcagtcagatctcagtactgagctctttctgagtcaagagaccttttcgtacttgggaaaactccttcctgagaagctggttctgtccccctcactgtccccagcagcggaggcagtagacgatctgctactcccagaagatgctgcagactggctagaaagccaagctggggctcaaggaatatcagcagcccctgcaccagccacccttacaccagccacctcctagacactttcatcctctgtcccttcccagaagacctactgcagcaactgtggtttccatcttggcttcctgcattctgggacagccaagtctgtcacctgcacgtactcccctgaccttaacaagctgttttgccagctggcaaagacctgtccagtgcagccgtagctcagctcaccaccccaccccaccccagcacctgtgttcacaccatggccatctaccagacgtcagcatatgacagaggtcatgcagcactgcccccaccttgagtgctactccgactatagtgatggcctggccgctcctcagcatcttatccaggtgggaggaatcctgcgtgctgatatttgtaggacaccattactcttcgacatagtgtgggttaccctatgagctacctcaggtcagttctgactaccaccatccacttcatgtgtagcagctcctgcatggggggggggaacccatcctcaccatcatcacactggaagactccgatggtaatctgctaggacacaacagtttcgaggtgcatatttgtactgttctgggagagacagatgtacagaggaagaaaatttccacaacaagtgggagccaccctctgagaggatcactaagtaagcactgcccaccagcaccagctcctctaccgagccaaagaagaagccagtggatgaaaaatatttcacccttaagatccgtgggcatgaatgcttcaagatgttcctagagttgaacgaggcattggagctgaaggatgcccaggctgggaagcagccagaggggagcagggctcaatgcagccttccaaactctaagaaaggggaatctaccacccactgtaaaaaactaatgttcaagagagaggggcctgactcagactga

SEQ ID NO: 34 - protein, 79 aa, synthetic construct; identical to SEQ ID NO: 6

SEQ ID NO: 35 - DNA, 1126 bp, synthetic construct
  source 1..1126: mol_type = other DNA
atggaggagcctcagtcagatctcagcactgagctccctctgagtcaagggacgttttcatacttggggaaactccttcctgagaagctggttctgttcccctcactgtccccagcagcagaggcagtagacgatctgctactcccagaagatgctgcagactggctagaaagccaagctggggctcaaggaatatcagaagcccctacactagccacctcctggacgctgtcatcctctgtcccttctcagaagacctacccagcacctatcatttctgtctgggcttcttgcattctgggacagccaagtctgtcacctacacgtactcccctgaacttaacatgctgttttgccagctggcaaaggcctgtccagtgcagctgtgggtcacctcaacacccccgcccagcacctgtgttcacaccatggccatctaccagacgtcagcatatgatggaggtcgtgaagcactgcccccaccttgagtgccgctctgactatagcgattgcttagaccctcctcagcaccttatccagtgggaggaaacctgcatgctgagtatttggaggacaccatcactctatgacatagtgtggggtgccctaggagccaccagaggtcggttctgactaccaccatccacttcaacttcatgtgtaacagctcctgcatggggggcaggaacccatcctcaccatcatcacactggaagactccaatggtaatccgctgggacacaacagttttgaggtgcatatttgtacttgtcctgggagacacagatatacagaggaagacaatttccataagaagtgggagccttgccctgagccaggcttggggaggatcactaagcgaacactgcccaccagcaccagctcctctaccaagccaaagaagaagccactggatgaaaaatacttcactcttcagatccatggccatgaatgcttcaagatgttcctaaagctcaacgaggccttggagctcaaggatgcccaggctgggaagcagccagaggggaacagggctcaatccagccttcccaagtctaagaaaaggcaatctacctcccgccataaaaaacttatgttcaagagagagcagcctgactcagactga

SEQ ID NO: 36 - protein, 210 aa, synthetic construct
MEEPQSDLSTELPLSQGTFSYLGKLLPEKLVLFPSLSPAAEAVDDLLLPEDAADWLESQAGAQGISEAPTLATSWTLSSSVPSQKTYPAPIISVWASCILGQPSLSPTRTPLNLTCCFASWQRPVQCSCGSPQHPRPAPVFTPWPSTRRQHMMEVVKHCPHLECRSDYSDCLDPPQHLIQWEETCMLSIWRTPSLYDIVWGALGATRGRF

SEQ ID NO: 37 - DNA, 1128 bp, synthetic construct
  source 1..1128: mol_type = other DNA
atggaggagcctcagtcagatctcagtactgagctctttctgagtcaagagaccttttcgtacttgggaaaactccttcctgagaagctggttctgtccccctcactgtccccagcagcggaggcagtagacgatctgctactcccagaagatgctgcagactggctagaaagccaagctggggctcaaggaatatcagcagcccctgcaccagccacccttacaccagccacctcctagacactttcatcctctgtcccttcccagaagacctactgcagcaactgtggtttccatcttggcttcctgcattgtgggacagccaagtctgtcacctgcacgtactcccctgaccttaacaagctgttttgccagctggcaaagacctgtccagtgcagccgtagctcagctcaccaccccaccccaccccagcacctgtgttcacaccatggccatctaccagacgtcagcatatgacagaggtcatgcagcactgcccccaccttgagtgctactccgactatagtgatggcctggccgctcctcagcatcttatccaggtgggaggaatcctgcgtgctgatatttgtaggacaccattactcttcgacatagtgtgggttaccctatgagctacctcaggtcggttctgactaccaccatccacttcatgtgtagcagctcctgcatggggggggggaacccatcctcaccatcatcacactggaagactccgatggtaatctgctaggacacaacagtttcgaggtgcatatttgtactgttctgggagagacagatgtacagaggaagaaaatttccacaacaagtgggagccaccctctgagaggatcactaagtaagcactgcccaccagcaccagctcctctaccgagccaaagaagaagccagtggatgaaaaatatttcacccttaagatccgtgggcatgaatgcttcaagatgttcctagagttgaacgaggcattggagctgaaggatgcccaggctgggaagcagccagaggggagcagggctcaatgcagccttccaaactctaagaaaggggaatctaccacccactgtaaaaaactaatgttcaagagagaggggcctgactcagactga

SEQ ID NO: 38 - protein, 79 aa, synthetic construct; identical to SEQ ID NO: 6

SEQ ID NO: 39 - DNA, 1121 bp, synthetic construct
  source 1..1121: mol_type = other DNA
atggaggagcccaagtcagatctcagtactgagctccctctgagtcaagagactttttcatacttggggaaactccttcctgagaagctggttctgtccccctcactgtccccagcagcggaggcagtagacgatctgctgctcccaggagatgctgcagactgcctagaaagccaaggtggggctcaaggaatatcagcagcccctgcaccagccacccttacaccagccacctcctggacactctcatcctctgtcccttcccagaagacctactgcagcaactgtggtttccgtcttggcttcctgcattctgggacagccaagtctgtcacctgcatgtactcccctggccttaacaagctgttttgccagctggcaaagacctgtccagtgcagctgtagctcagctcaccaccccaccccagcacctgtgttcacaccatggccatctaccagacgtcagcatatgacagaggttgtgcagcactgcccccaccttgagtgctgctccgactatagcgatggcctggccgctcctcagcatcttatccaggtgggaggaatcctgcgtgctaatatttgtaggacaccatcactcttcgacatagtgtggggtaccttatgagctacctcaggtcggttctgactaccaccatccacttcaacttcatgtgtagcagctcctgcgtgggggcgggaaacccatcctcaccatcatcacactggaagactccgatggtaatctgctaggacacaacagtttcgaggtgcatatttgtactgttctgggagagacagacgtacagaggaagaaaatttccacaacaagtgggagccaccctctgagaggatcactaagtaagcactgcacaccagcaccagctcctctactgagccaaagaagaagccagtggatgaaaaatatttcacccttaagatccgtgggcatgaatgcttcaagatgttcctagagttgaatgaggctggaggatgcccaggctgggaagcagccagaggggagcagggctcaatgcagccttccaaactctaagaaaggggaatctaccacccactgtaaaaaactaacgttcaagagagaggggcctgactcagactga

SEQ ID NO: 40 - protein, 134 aa, synthetic construct
MEEPKSDLSTELPLSQETFSYLGKLLPEKLVLSPSLSPAAEAVDDLLLPGDAADCLESQGGAQGISAAPAPATLTPATSWTLSSSVPSQKTYCSNCGFRLGFLHSGTAKSVTCMYSPGLNKLFCQLAKTCPVQL

SEQ ID NO: 41 - DNA, 1120 bp, synthetic construct
  source 1..1120: mol_type = other DNA
atggaggagcctcagtcagatctcagcactgagctccctctgagtcaagggacgttttcatacttggggaaactccttcctgagaagctggttctgtccccctcattgtccccagcagcagaggcagtagatgatctgctactcccagaagatgctgcagactggctagaaagccaagctggggctcaaggaatatcagaagcacctacactagccacctcctggacgctgtcatcctctgttccttctcagaagacctacccagcacctatcatttctgtctgggcttcttgcattctgggacagccaagtccgtcacctacacgtactcccctgaacttaacatgctgttttgccagctggcaaaggcctgtccagtgcagccgtgggtcacctcaacacccccgcccagcacctgtgttcacaccatggccatctactagacatcagcatatgatggaggtcgtgaagcactgcccccactttgagtgccgctctgactatagcgattgcttggaccctcctcagcacctcatgcagtgggaggaaacctgcatgctgagtatttggaggacaccatcactctatgacatagtgtggggtgccctaggagccaccacaggtcggttctgactaccaccatccacttcatgtgtaacagctcctgcatggggggcaggaagccatcctcaccatcatcacactggaagactccaatggtaatccgctgggacacaacagtttcgaggtgcatatttgtacctgccctgggagatgcagatgtacagaggaagacaatttccacaacaagtgggagccttgccctgagccaccctctgggaggatcactaagcaaacactgcccaccagcaccagctcctctaccaagccaaagaagaagccactggatgaaaaacacttcacccttcagatccatgggcatgaatgtttcaagatgttcctaaagctcaacgaggccttggagctgaaggatgcccaggctgggaagcaaccacaggggagcagggctcaatgcagccttcccaagtctaagaaaaggcaatctatctcccgccataaaaaaataatgttcaagagagagcagcctgactcagactga

SEQ ID NO: 42 - protein, 210 aa, synthetic construct
MEEPQSDLSTELPLSQGTFSYLGKLLPEKLVLSPSLSPAAEAVDDLLLPEDAADWLESQAGAQGISEAPTLATSWTLSSSVPSQKTYPAPIISVWASCILGQPSPSPTRTPLNLTCCFASWQRPVQCSRGSPQHPRPAPVFTPWPSTRHQHMMEVVKHCPHFECRSDYSDCLDPPQHLMQWEETCMLSIWRTPSLYDIVWGALGATTGRF

SEQ ID NO: 43 - DNA, 1604 bp, synthetic construct
  source 1..1604: mol_type = other DNA
atgacgtgcctttaaaatcctacattaaggaattacccagagaatttgcttggccccagccctgcagacaaaacatgttttatgtcctgatggcaggtgaagggaggcagcactgagaactggaagatccagatgagatccaagcttcagtcttagtccccttgttttctgccattaactagccctgttagcttggtcaggtcacctagtttctgaattgaaacttcctgtctataaaatgaaaagtttgaattaaaccatctctggaattctttctagctatgactttgtgattctaagataatgcataactatctggaagcaatgttaacgagtcaaaagcccgaagccaccatcctgggcacagctagctgctgggctctgggggcaccttctttctggacttggagagtccttcccaagaaggttacacacttccctgaggttccgtagccagactgccttccctggagctgcagtggaggagcctcagtcagatctgagcattgagctccctctgagtcaagagacattttcatacttggggaaactcctttctgagaagctggttctatccccctcactgtccccagcagcggaggcagtagtcaatctgctactcccagaagatgctgcagactggctagaaagccaagctggggctcaaggaatatcagaagcccctacactagccacctcctggacgctgtcatcctctgtcccttctcagaagacctacccagcacctatcgtttctgtctgggcttcttgcattctgggacagccaagtccgtcacctacacgtactcccctgaacttaacatgctgttttgccagctggcaaaggcctgtccagtgcagccatgggtcacctcaacacccccgcccagcacctgtgttcacaccatggccatctaccagacgtcagcatatgatggaggtcgtgaagcactgcccccaccttgagtgccgctctgactatagcgattgcttggaccctcctcagcacctcatgcagtgggaggaaacctgcatgctgagtatttggaggacaccatcactctatgacatagtgtggggtgccctaggagccaccagaggtcggttctgactaccaccatccacttcaacttcatgtgtaacagctcctgcatggggcgcatgaacccattctcaccattatgacaatggaagactccaatggtaatccgctgggacacaacagtttcgaggtgcatatttgtacctgtcctgggagacacagatgtacagaggaagacaatttccacaacaagtgggagccttgccctgagccaccctctgggaggatcactacgcaaacactgcccaccagcaccagctcctctacgaagccaaagaagaagccactggatgaaaaatacttcacccttcagatccatgggcatgaatgtttcaagatgttcctaaagctcaatgaggccttggagctgaaggatgcccaggctgggaagcagccagaggggagcagggctcaatctagccttcccaagtctaagaaaaggcaatctacctcccaccataaaaaactaatgttcaagagagagcagcctgactcagactga

SEQ ID NO: 44 - protein, 59 aa, synthetic construct
MMEVVKHCPHLECRSDYSDCLDPPQHLMQWEETCMLSIWRTPSLYDIVWGALGATRGRF

SEQ ID NO: 45 - DNA, 2212 bp, synthetic construct
  source 1..2212: mol_type = other DNA
atgaaacacataggattaaaaaggaagccatgtggagaaattagaatcctggtgcgttgctggtggactgtaaaacggcagtcactgtggaaaacagttgggcagtttctcaaaacgttcaacatggaattaccatatgacccagcaattccactcctaggtatatacccaaaatatctgaaagcaggcactcaaacagatacttgcagacaaattttcaaggcagcattattcacaacagccaaaagacagaaacaacccaagtgtccaccaacggatgataaacagaacctggaacatacacatgatggactattattattcggccattaggaaaaatgacgttcagatacatgctaccacatggatgacccctgaaaatattagcccttagggagttcatgaacttcaatttaagaacaagtgattggggaaggactcttctgggatggtcaaagttttttcttacaggaaacagacgatgtgcagaaaagttttaaaaacccaaagatgcacagaaaaatcctgctcgggatcagtcagggaagaaaagaatgagctgcaaagagaagtctaaccttgactttctccccaaacccaacgctattcatgacgtgcctttaaaatcctacattaaggaattacccagacaatttgcttggccccagccctgcagacaaaacatgttttatgtcctgatggcaggtgaagggaggcagcactgagaactggaagatccagatgagatccaagcttcagtcttagtccccttgttttctgccattaactagccctgttagcttggtcaggtcacctagtttctgaattgaaacttcctgtctataaaatgaaaagtttgaattaaaccatctctggaattctttctagctatgactttgtgattctaagataatgcataactatctggaagaaatgttaacgagtcaaaagcccgaagccaccatcctgggcacagctagctgctgggctctgggggcaccttctttctggacttggagagtccttcccaagaaggttacacacttccctgaggttccgtagccagactccttccctggagctgcagtggaggagcctcagtcagatctgagcattgagctccctctgagtcaagagacattttcatacttggggaaactcctttctgagaagctggttctatccccctcactgtccccagcagcggaggcagtagtcaatctgctactcccagaagatgctgcagactggctagaaagccaagctggggctcaaggaatatcagaagcccctacactagccacctcctggacgctgtcatcctctgtcccttctcagaagacctacccagcacctatcgtttctgtctgggcttcttgcattctgggacagccaagtccgtcacctacacgtactcccctgaacttaacatgctgttttgccagctggcaaaggcctgtccagtgcagccatgggtcacctcaacacccccgcccagcacctgtgttcacaccatggccatctaccagacgtcagcatatgatggaggtcgtgaagcactgcccccaccttgagtgccgctctgactatagcgattgcttggaccctcctcagcacctcatgcagtgggaggaaacctgcatgctgagtatttggaggacaccatcactctatgacatagtgtggggtgccctaggagccaccagaggtcggttctgactaccaccatccacttcaacttcatgtgtaacagctcctgcatggggcgcatgaacccattctcaccattatgacaatggaagactccaatggtaatccgctgggacacaacagtttcgaggtgcatatttgtacctgtcctgggaaacacagatgtacggaggaagacaatttccacaacaagtgggagccttgccctgagccaccctctgggaggatcactaagcaaacactgcccaccagcaccagctcctctacgaagccaaagaagaagccactggatgaaaaatacttcacccttcagatccatgggcatgaatgtttcaagatgttcctaaagctcaatgaggccttggagctgaaggatgcccaggctgggaagcagccagaggggagcagggctcaatctagccttcccaagtctaagaaaaggcaatctacctcccgccataaaaaactaatgttcaagagagagcagcctgactcagactga

SEQ ID NO: 46 - protein, 59 aa, synthetic construct; identical to SEQ ID NO: 44

SEQ ID NO: 47 - DNA, 1127 bp, synthetic construct
  source 1..1127: mol_type = other DNA
atggaggagccccagtcagatctcagcactgagctccctctgagtcaagagaccttttcatacttggggaaactccttcctgagaagctggttctgtccccaccactgtccccagcagtggaggtagcagacgatctgctactcccagaagatgctgcagactggctagaaagccaagctggggctcaaggaatatcagtagcccctgcaccagcctctcctgagccagccacctcctggacactgtcatcctctgtcccttctcagaagacctaccccagcatctatggtttccatctgggcttcttgcattctgggacagccaagtccgtcgcctacatgtactcccctgaccttaacaacctgttttgccagctggcaaagacctgtccagtgtagccgtgggtcacctcaccaaccctgcccagcacctgtgttcacaccatggccatttaccataagtcagcatatgacggcggtcgtgcagcactgcccccaccttgggcgctgctctgactatagcgatggcctcgtccctcctcagcacctcatccagcggggagaaaacctgcgtgctgagtatttggaggacactagcactctttgacatagtgtggggtgccctatgagccaccagaggtcggtctgactaccaccatccacttcatgtgtaacagctcctgcagcaacccatcctcaccatcatcacactggaagactccaatggtaatctgctgggatgcaacaggttcgaggtgcatatttgtacctgtcctgggagaggcagatgtatagaggaagacaatttccacatgaagtgggagccttgccccgagctaccctctgggtggatcactaagcgaatgctgcccaccagcaccagctcctctaccaagccaaagaagccgccactggatgaaagatatttcacccttcagatccgtggacatgaacgctacaagatgttctagagctgaatgcggccttggagctgaaggatgccgaggctgggaagcagccagaggggagcagggctcaattcagccttcccaagccttagaaagggcaatctacctcccaccataaaaaaacaaacattcaagagagaagggcctgactcagactaa

SEQ ID NO: 48 - protein, 132 aa, synthetic construct
MEEPQSDLSTELPLSQETFSYLGKLLPEKLVLSPPLSPAVEVADDLLLPEDAADWLESQAGAQGISVAPAPASPEPATSWTLSSSVPSQKTYPSIYGFHLGFLHSGTAKSVAYMYSPDLNNLFCQLAKTCPV

SEQ ID NO: 49 - DNA, 1130 bp, synthetic construct
  source 1..1130: mol_type = other DNA
atggaggagcccgagtcagatctcagtactgagctccctctgagtcaagagacttttttgtacttggggaaactccttcctgagaagctggttctgtccccctcactgtccccagcagcggaggcaggagacgatctgctactcccagaagatgctgcagactggctagaaagccaagctggggctcaagaaacatcagcagcccctgcaccagccacccttataccagcctcctcctggacactctcgtcctctgtcccttcccagaagacctactgcagcaactgtggtttccgtcttggcttcctgcattctgggacagccaagtctgtcacctgcatgtactcccctgaccttaacaagctgttttgccagctggcaaagacctgtccagtgcagccgtagctcagctcaccaccccaccccagcacctgtgttcacaccatggccatctaccagacgtcagcatatgacagaggtcatgcagcactgcccccaccttgagtgctgctctgactatagcgacggcctggccgctcctcagcatcttatccaggtgggagaaatcctgtgtgctgatatttgtaggacaccatcactcttcgacatagtgtggggtaccctatgagctacctcaggtcggctctgactaccaccatccacttcaacttcatgtgtagcagctcctgcaagggggggaggaacccatcctcaccatcatcacactggaagactccagtggtaatctgctaggacacaacagtttcgaggtgcatatttgtacctgttctgggagagacagacgtacagaggaagaaaatttccacaacaagtgggagccaccctctgagaggatcactaagtaagcactgcccaccagcaccagctcccctaccgagccaaagaagaagccagtggatgaaaaatatttcacccttcagatccatgggcatgaatgattcaagatattcctagagttgaatgaggcactggagctgaaggatgcccaggctgggaagcagccagaggggagcagggctcaatgcagccttccaaactctaagaaaggggaatctaccacccactgtaaaaaactaatgttcaagagagaggggcctgactcagactga

SEQ ID NO: 50 - protein, 134 aa, synthetic construct
MEEPESDLSTELPLSQETFLYLGKLLPEKLVLSPSLSPAAEAGDDLLLPEDAADWLESQAGAQETSAAPAPATLIPASSWTLSSSVPSQKTYCSNCGFRLGFLHSGTAKSVTCMYSPDLNKLFCQLAKTCPVQP

SEQ ID NO: 51 - DNA, 1583 bp, synthetic construct
  source 1..1583: mol_type = other DNA
atggcacctttctactggcttccaggaatcctgtcactgctgcttgttctgcactgggaatatggggcggggaacccccttcccaacactcctgacaaggccacctgcatcactgaccacccatgtgccagcaacctcatgacccagatcaagaaccaactggaacagctcaacagtagcgccaacaccctccttgttctctacgtaagataccctttcccatacccaggtaccaaggaaggaggggtggccctggggtgacaggagtgggcagaattcctgcaggtatcagtagaggctgtgctgaatttggaggggaagaggggtagctgatcacggaggggcccttgaagctagaaggaatgaagggggcctggttaaaccacacccagcttttcccctacccaacccccagcccaaactagaatggactttcccagggttagattggtccagagctggggtcagaaagtatgggccctggcacttcaggtattaagattcctccaccacttggagcaagaggcagatagagatacacaaaggaggcaaaagaaagggaagtgttcagagaagtgctgttgagtgagatggtgggtgatgggtgtctgagtgcagagagacacccagataggtgaaaggacaagtgtctatggcacagctctgaaggtgaggcatccaggttttctttcttttcccatattatcatgataattacaaaaaaaaaggtgtaatcagtttgattctgactcatggcaccccatgtgtgtcagaatagaattgtattctgtaggttttgcaatggctgatttttgcaagtagattgcagtacctttctttcaaggcacttctgggtggtctcaaacctccaaccttttcaattagcagccaaggacattaaccatttgcaccacccagggactcttaacactaagtaacaataacaattcttgcatgatagaggccttgattctttaccaagtttgttcacacttggatgtccatttgcctttcatgactatcctgagaggtctacagggcacagagttgggtcctcatgccataggtggggaaacagaggcccaagagcttgcccctagggttggtggcagggctggaacactgacccttgcctccttgctcacctccctctgccctctgttcctcagtatactgctcagggcgagccattccataaagaagagggcaagctgtgtggtggccagaacttgacaaatttcccacccttccacaccaacggcacagagaaggacaagctggtagagatctaccaaatcattgcctactttaacacctccctgggtagcatcatacaggaccagaagaccctcaaccccgaagaccggaacctccacagccagctggactccatagtggacaccctggaggggctcctcagcaatgtgttctgccacctgtgcaatgagtaccaagtgggccaagtggatgtgtattacagcccctacacctcagccaaagacatcttcaagaagaagaagctgggctgtcaactgctggggaagtatgagcaggtcattgctgagctggcccaggacttctag

SEQ ID NO: 52 - protein, 86 aa, synthetic construct
MAPFYWLPGILSLLLVLHWEYGAGNPLPNTPDKATCITDHPCASNLMTQIKNQLEQLNSSANTLLVLYVRYPFPYPGTKEGGVALG

SEQ ID NO: 53 - DNA, 1664 bp, synthetic construct
  source 1..1664: mol_type = other DNA
atgacccagatcaggagtcaaatgggccagctcaacagtagtgccaacgacgtctttattgcctatgtaagttacccctctaaaccttggggaccgaggaaggagggtgtgggcctgaggtgggagaagtgggcagaatcactgcaggtatcactagaggccgcactgacctgagctgggggtgctggccagggaggacaccttttgtagctggaaggaaagaaagggggctagttagaccacacccggctttcccaacatcttaccatctgcacagatgggaattgactttcccagggttaggtcagtgtagagctctggtgagaatgtaaatggcctggcacttcaggtttgggtgttccccgtcacagtggagtgagaggcagatggagatgcacaaaggagggaaaagtcagggaggtggtcagagaggtaactttgagggagatggtgggctgtgggtatctgggtacaagagatatccaggtaggtgaaaggccaatgtgtgacacacctctgaaggtgaagcaccctggttttctttctttctccatatgatcacgataattaaaaataaatcaaaactagttgccaccagtcaattctgactcacggcaacccgatgtgtgtcagaggggaactgtaccccatagattttgcagtggctgagtttctggaagtagatcaccaaactttctttggaggtgcctccgggtggactcaaacttcagaacttttaattagaagccgagtgccttaaccttttccaccaccaagggactcctaatactaagtggtaataactgtccttgaatgccacaggccttgattcttctcactcatttatttactttttattgtgctttaggtgaaagtttatagctcaagttagtttctcatacaaaactttatacacacattgttatgtaagcctagctgctatctgtgtaatgtgacggcatactcctcctttccaccccagattgtccctgtccattcaaccagctcttagccctgtctaccttcttatctctcctctggacaggaacttgattctttaccaagtgggttcacacccagatctccacctgcctctcatgaggatcctgatttgtgtatagggcacagagttgggtctccatgccataggtagggaaactgaggcccaatagcttgccacaagagtaggtgccagggctgaaacaccgatccctgcctccttgctcacctcccctgcaccctctgcttctcagtagatgggccagggccagcctttctccagcgatgtggaccacatgtgcggccatggcttgatgagcttcccacccttccacgccaatggcatcaagagagagaagctggtggaactctaccgcatcatggtctactttgacttctccctggatgacaccctcaagttccagaagaccctctaccccaatgatcagaacctccacagcaagctcaagtccacgatggacatcctaccggggctcctcagcaacgtgctctgccgcctgtgtagcaagaacaacatgaggctgacggacacaatctatggcctcaacatctcacaggagaatgtcttccacaagaagaagctgggctgtgagctcctgttgaagtacacacagctcatcccctatctcgcctgggacttctag

SEQ ID NO: 54 - protein, 57 aa, synthetic construct
MTQIRSQMGQLNSSANDVFIAYVSYPSKPWGPRKEGVGLRWEKWAESLQVSLEAALT

SEQ ID NO: 55 - DNA, 1805 bp, synthetic construct
  source 1..1805: mol_type = other DNA
atgacgtctttcggcagactcctaggaatcctgtcactgctgcttattctacattgggaatatgggacagggagccccctttgcaacactcctgacaatgccccctgtgtcttggaccacccatgtgccagcaacatcatgacccagatcaggaaccaactggcgcagctcaacaggagtgccaaggacctctttattgcctttgtaagttacccctccaacccctggggaccaaggaatgagagtgtgggcctgaggtgggagaagtgggcagaatccctgcagatatcaggagagtctgcactgacctgagcagggggtgctggccagggaggagaactttttgtagctggaagggaagaaaggggactggttagaccacacccagctttccccacttcctactacctgcacagatgggacttaactttcccagggttaggtcagcttagagctctgatgagaatgtaaagggcctggcgcttcaggtttggggattcccccatcacagtggggcaagaggcagatggagatgcacaaaggagggaaaaggcagagggtggtcagagaggtgcctttgagcacgatagtgggcggtgggtgtctgggtgcagagaattcataggtgggtgaaaaggcaatgtgtgtgacacacctctgaaggtgaggcaccctggttttctttctttctccatatgatcacgataattaaatataaatcaaaattagttgccaccagccgagtctgactcagccaacactgtgtgtgtcagagtgaaactgtaccccacaggttttgcaatcgctgattttttgcaagtagatcagcaaatctttcttccgaggctcctcaaggtggactcaaacttccaacctttcaattagaagccgagtgccttaaccttttccaccacccagggattcctaatactaagtagtaataactgtccttgaatggcagagaccttgatacttttcattcatttatttactttttattgcactttaggtgaaagtttaaagctcaagttagtttctcatacaaaactttatacacacattgctatgtgagcctagctgctatccccacaatgtgacagcatattcctcctctccaccccagatttcacctgtccattcaaccagtgcttagccctttctgcctgctcatctcacctctggacaggaacttgattctttaccgagtgggttcacatccagatttccacctgcctcttatcaggatcctgatttgtatatagggcacggagttgggtctccatgccataggtagggaaactgaggcccaatagttggctggaggagtgggtggcagagttgaaatactgatcactgcctccttgctcacctcccctctgccctctgtttcttagtccaaggcccagggcgagccattccccagtgatatgcgtaagatatgcagccacggctttattaagttcccacctttccatgccaacggcacggaaaaggacaagttggtggagctctacagcatcacggtcttctttgtcttctccctggatgacatcaccaagttccagaagaccttcatccgcaaggatcagaacctccacagcaagcttaagtctacgatggacatcttggcagggctccgcagcaacatgctctgccgcctgtgcagcgggcacaacatgaggccgacggacccaatctctggcctcgacatctcacagaaggactggttccacaagaagaagctgggttgtgagctcctgataaagtacgcacggctcatccctgtgctagcccgggacttctag

SEQ ID NO: 56 - protein, 103 aa, synthetic construct
MTSFGRLLGILSLLLILHWEYGTGSPLCNTPDNAPCVLDHPCASNIMTQIRNQLAQLNRSAKDLFIAFVSYPSNPWGPRNESVGLRWEKWAESLQISGESALT

SEQ ID NO: 57 - DNA, 4305 bp, synthetic construct
  source 1..4305: mol_type = other DNA
atggaaatcagtgtggaacgcacccatcttccgggtaccctgttacaacttctctcagctcacctttgcagaaaggcttcaagtgtggctgttagcaacccctctgtgttacaagtccacttttttttttttttaattccttgtatacttttggtgaaagtttacataacaaattaggttccactttaacaatttctatacataccgttcagtgtcatcgattacgtttttcacaatgtgtcacaatgctcattacttccattctggttgttctgtttctattaatctagcttccctgactccccttacttttttgtctttgctttagggtgaatattgactgtctgttctcatacagatggttatttaaaagaagcacagtacacatgggtgatatggcttattttatgagccagtctattatttggctgaaaggtgatctccaggagtgggttcagttccaaatttaaaggctatctcagggtgatagtctcgggggatcctctagtctctacttgtccagtaagtctggctttttttttttttttaaggaatttgagtttttttctatgtttttattccattctacccggggctgaccatctattgtgtccctgttcagaatggttgttagtggtagccaggtaccatctagttcttctggtctcaagatagatgaggctgtggtttgtggaagctattagtcctgtagactggtttcttctttgaatctttggtttccttctttctcttttgctccagatgagtacagaccaatagttgtatagcaatccctcttgatgggtgtccaaagtccttagctctttcatagctgtggtgggggtggtggccagtcagcttctgtgatggttaaggttgtgtgtcgacttggctgggccatgattctcagtggttggcagttatgatgtagtttggcagtcatataatgttgtgatcatttccatgatgagatttgatataatgtgaatacctccatgatgggatctgctgtgagtagctgaccagttgaaagggagtttctttgcgtgtgttgcttgcattgactataagtggacattctggcaaggcttatgggcttttgctcactctggatcttgcagctggctcctgttcatgacttctgttctggggacttgagctagcagcttacctgtggtcttgcctgctgatctagggatttgttcatgtttgaagactatgaccaagaccctactctcatacctgctgaccttgagttcgacagtcctttgactatgtgaatcaggaagagcttctatcctgacccatgaacttgggacactccagcctctacaactgtgtgagccatttccttgatataaatctctgtctatataaatttacaggctttatttttttgcttctctagagaacccagcctaagacagctttcgttgaccattataccccttgatgtagtgatcttccagactctgatgtacatgctccaaacgtacatgcagaacaccacgtagcagtcagtgcacacctttgtgcatggggctgtaaactgagaacatgccgtggagtacttctttgaagttctgggaaacagggaagaaagcctttaaataagtgacacctgctggggagaaaggcatgtggattacctgggaggcatgcatagcttcctgatgacataagctgtactggagatctaaaagggaaggcctgactctcctttcctggaaacaagaaaaatgtaaattacatggcactttcgaaaccctggtggcagagtagttaagagcttggctgctaaccaaaaggttggcagttcaaatctaccagctgctccctgaaaactctgtggggcagttctactttgtcctatagggtaactgtggattgcaatcgggttttctgtttccttttccatgttgatattcatcatttaagaccttgattgttcaccttttttcaagtgactagtgcagctactttccatcttgttcatatggcggcatagtcttttactcaagttctcagttattacaagattttgtctctttatcatatatgctttcctacgcatatgtgaatggtaaaggactctttgaggtgcaatctgagccaagacatggaggggatgaaattgagagacctaggtaggggagtggctacttttagggaaggtcagagatggaggagatgggatggtacgtattgggggcagttggcctggtaaaccataaacacgtggcagatttggggggaagaagagggtcacaaaaggacccctgcctcccagctgaggttcccacggaagcccagatttggattgctttggggagtcgaatgtaaaacaatggttaggaatttaagtcatcctgaagatggggaaataagggtctcccattcctttgaagcaaacatttccccctgggctggggagtggtaacctcaagcaaggagctgggtggcttttgccttcttgagggaaaggattggatgtttggatgtcatcctagaccaagcttccaatccgagggagtagctcgagtcctgcccccttaccgcttcttttaaggcaggagagagcaagggggctttaacaaggtggagagaagcagggtaggtagaatttgagatgcagagaagccaggagtcccagggagttgaagtgcatccagtgtgacctgaaagcttaaactgcgatccccaccaccctgaccatggagcttttctcttggtttccaggagtcttgctgctgcttgttctgcaccaggaacatggggcaaggagccttgataccgtcactcctagcaaggtgacctacaccacatgtcacacatgtcctggcaacctcacgacccagatcaggaacaaactgaaacagctcaacggtagtgctgacggcctcctaactctctatgtaagatattccttcccatccctagggacagaggaaggaggggatgggcctgggttggcaggagtgggggaattcctgcaaacatcagtagaggctgcagggaactgggctagagaagggggctggccagggaagctggaagcagagaagtttgtgcggttaaaccgcaccctacccacagctccgactggaatcgactatcacagggttaggtcggggcagggctctggtgtgaatggcacttcaggttctgggattcctccaccaagtggagcaagaggcagatggagatacacaaaggagggaaaatgggaggaggtgttctgagaggtgccattgagagagactggtgggcggtgggtgcctgggttgcgagagatgcccagatgggtaaaagggcaagtgagagggaccctggctttctttctttctctgtatgatcatgacagctaaaaatgaaacataatccgttgttgccagttgattctgactcgtggcaattccacgtgtgtcagagtagaactgtactccatagcctttgtaatggctgattttttgcaagtagatcacccagcctttttcttgaggtgcctctgggtggactagaacctccaatctttcaattagcagcctagggtgttaacaatttgtaccacccagggactcctaacactaagtaacagtaacaatcgttgcatggtaaaggccttgattctttaccaagtgtgttcacacccagatgtccatctgcctctcatgacgattctgagatgcctacagggtacagagttgggtcctcatggcataggaggggaaactaaggcccaagagcttgttgtaagggtgggaaacagggctggaacaaggattgctgcctccttgttgacctctcctctgccctttgctcctcagtgcatcgcccaaggggagccgttccccagtgaagtggacaagatgtgtggcagtggcttgacagacttcccacccttcaatgctcatggcaggaaggaggccatgatgaaagagctgtactgcatcgtcacgtacctaactacctccctgggcaacatcacacgggaccagaagtccctcaacccgcaagaccggaacctccatggacagctagacttcgtggcaaccattatgcagcagctccagagcaacgtgctctgcctcctgtacaaggacaaagtggaccacgtgggggaatgctacgggcccaacaccttgggcaaaaccttcttccagaagaagcggctgggctgtcaggtcctgtttaagtacaagcaggtcatcactgagctgtatcaggccttctaggagcaggatcttgaagtgtgctgtgtggattaagggatcttgggagtgggatctagaggtggtgagcact

SEQ ID NO: 58 - protein, 175 aa, synthetic construct
MELFSWFPGVLLLLVLHQEHGARSLDTVTPSKVTYTTCHTCPGNLTTQIRNKLKQLNGSADGLLTLYVRYSFPSLGTEEGGDGPGLAGVGEFLQTSVEAAGNWAREGGWPGKLEAEKFVRLNRTLPTAPTGIDYHRVRSGQGSGVNGTSGSGIPPPSGARGRWRYTKEGKWEEVF

SEQ ID NO: 59 - DNA, 1497 bp, synthetic construct
  source 1..1497: mol_type = other DNA
atggcaccttttggccagttcccaagaatcctgtcattgctgctttttctgcactggaagcgtggggcagggagcccccttccctgacaatgacacctgcaccacatcacccatgttccagcaacctcatgacccagatcaaaaaccaactggtatagctcaacagtagtgctaaggaccactttattgcctatgtaagttaccccttcccacccccggggaccaaggaaggagggtgtgggcctgaggtgggagaagcaggcagaatccctgcaggtatcatagaggctgcactgacctgagctgggcatgctggccagggaggagaccttttgtagctggaaggaaagaaggggaggtggccacacccagctttccccacatcctacccctagcccagacaggaatcgactttcccagggttaggtcagagtagagttctggtgagaatgtaaagggccaggtgcttcaggtttgggggttcccccatcacagtggggcaagaggcagatggagatgtacgaaggagggaaaaggcaggcaggtggtcagagaggtgctgttgaaagagggtgggtggtgggtctgggtgagagagatacccagatgggtgaaagggcaatgtgtgtcacacacccctgaaggtgaggcaccccggttttctttctttccccatatgatcatgataattaaaaataaatcaaaaccagttgccactagttgattctgactcatggcaaccccatgtgtgtcagagtggaactgtaccccataggttttgcaagggctgattttttgcacgttagatcaccaaacgtttctttcaaggctcctccaggtgaactcaaacttccaacctttcaaatagcagccgagggcattaactatttctaccacccagggactcctaatactaagtaatgataataatccttgcatggtagaggacttgattctttaccaagtgtgttcacacccatggagttgggtctccatgccataggcggggaaattgaggcccaatagcttgctgcaagtgtggatggcaggactggaacaccgatccctgcctccttgctcacctcccatctgccctctgatcctcagtacgtggccaagggtgagccgttccccagcaatgtgggcaagctgtgcagccacagcttaatgaacttcccacccttccatgtgaatggcacagagagggaaaagctagtggagctgtaccacatcatggacaaaatcgtgaagtttcagaaggccctccaccccaaagataagaacctccacaggaagctaagctccatagcagacaccctgaggggactcccaaacaaaatactctgccacctgtgcaaggagtaccacgtgagcctggtggacatgacctacagcctcagcacctcagagagggatgtctcctagaagaagcagctgggctgtcagctcctgatgaagtacaagcaggccatggttgagctggcccacaacttctag

SEQ ID NO: 60 - protein, 28 aa, synthetic construct
MAPFGQFPRILSLLLFLHWKRGAGSPLP

SEQ ID NO: 61 - DNA, 4207 bp, synthetic construct
  source 1..4207: mol_type = other DNA
atggcatctttctaccggctcccaggaatcctctcactgctgcttgttctgcactagaaacatggggcacggagcccccttcccaacactcctgacaaggtcacctgtaccacagtcacccatgtcccaccaacctcactacccaggtcaggaatcaactgctgcagctcaaccgtagtgtcagctccctcctcattctctatgtaagttaccccttcccatccctggataccgaggacaggggaggtggggctggggtgacaggagtgggcagaattcctgcagataccagtagaggctgcactgacctaggctggtggagggctggccagggaggagacctttttggagatggaaggaaagaagaggaactggttagaccacacccagctttcccaacaccctacccctagccctttccaaggtcagaaagcacttcaggtgttaagattccttcactatgtggagcaagaggcaggtggagatacacaagggagggaaaagcagggacgtgtttagagaggtggcatggggaaggactgcgggcggtggatgtctggaagtgagggatgccaccgacagagcaaccccgtatgtgtcagagaagaaatgtactccatatgttttgcaatggctggtatttcgcaagtggatcaccaaagctttcttttgaggcacttctatgtgcactcgcctttcaattagcagccaagggcgttaaccatgtgcaccacccagggactactaatgttgttgttgttaggtgccgcagagtaggttccgactcatagcgaccctatgcacagcagaacgaaacactgcctggtcctgcgccatccttacaatccttgttatgctcaagctcattgttgcagccactgtgtcaatccacctcgttgagggtcttcctcttttccgctgaccctgtactctgccaagcatgatgtccttctccagggactgatccctcctgacaacatgtccaaagtatgtaagacgcagtcttgccgtccttgcttctaaggagcattctggttggacttcttccaagacagatttattcgttcttctggcagtccatggtatattcgatattctttgccaacaccaaaattcaaaggcgtcaactcttcttcggtctcccttattcattgaccagctttcacacaccatatgatgcgattgaaattaccatggcttgggtcaggcgcaccttagtcttcagggtgacatctttgctcttcaacactttgaagaggtctttgcagcagatttacccaatgcaatgcgtcttttgatttcttcactgctgcttccatggctgttgattgtggatccaagtaaaatgaaatccttgacaacttcagtcttttctccatttatcatgatgttgctcattggtccagttgtgaggatttttgttttctttatgttgaggtgtaatccatactgaaggctgtggtctttgaccttcattagtaagagcttcgagtcctcttcactttcagcaagcaaggttgtgtcatctgcatatcgctggttgttaatgagtcttcctcctatcttgatgcctcgttcttcttcatatagtccaggatcttggattatttgttcagcatacagattaaataggtatagtgaaagaatacaaccctgacatacaactttcctgactttaaaccaatcagtatccccttgttccgtctgaacaactgcctctggatctatgtaaaggttcctcatgagcacaactaagtgttctggaattcccattcttcgcaatgttaatccatagttagttatgatccacacagtcgaatgcctttgcataatcaataaaacacagataaacatccttcaggtattctctgctttcagccaggatccatctgacatcagcaatgatatccctggttccacgtcctcttctgaaaccggcctgaatttctggcagatccctgtcgatatactgctgcagccatttttgaatgatcttcagcagaattttgcttgcttgtgatatcgttctataatttccacattgggttggatcaccttccttgggaataggcagaagtaaggatctcttccagtcagttggccaggaagctgtcttccctatttcttggcatagacgagtgagcacctccagcactacatctgtttgttgaaacatctcaattgatattccatcaattcctggaggcttgtttttcgccaatgccttcagagcagcttggacttcttccttcagtaccatcggttcctgatcatataccacctcttgaaatgttcgaatatcaactaattctttttggtataatgactctgtgtattccttccatcttcttttgatgcttcctgcatcgtttaatattttcgccatggaatccttcactattgcaactcgaggcttgaattttttcttcagttctttcagcttgagaaacgccgagcatgttcttcccttttggttttccatctccggctctttgcacatgtcattataatactttactttgtcttctcgagaggtcctttgaaatcttctgttcagttcttttacttcatcaattcttccttttgctttagctgctccacgctcaagggcaagtttcagagtcttctctgacatccaccttggtcttttctttctttcctgtcttttcagtgacctcttgctttcttcatggatgacgtccttgatgtcattccacaactcatcttgtcttcagtcactagtgttcaatgtgccaaatctattcttgagatggtctctaaattcaggtgggatatattcaaggtcatattttggctctcgtggactcgctctgattttcttcagtttcagcttgaacttgcatatgagcaattgatagtctgttccacagtcggcccctggccttgttctgactgatgatattgagcttttccattgtctctttccacagatgtggtcaatttgatttctgtgtgttccatctggcaaggtccatgtatatagtcgccattcctgttggtgaaagaagatatttgcaatgaagaagtcattggtcttgcaaaattctatcatatgatctctggcattatttctatcaccaaggccatattttccaactactgatccttcttctttgtttccaacttttgcattccaatcgtcagtaattatcagtacatcttgattgcatgtttgatgaatttcagactgcagcagctggtaaaaatcttctatttattcatctttggccctagtggttggtgcgtaaatttaaatagtagttgtattaactggtcttccttgtaggcatatggatattatcctatcactgacagcgttgtacttcaggatagatcttgaaacgctctttttgacaatgaatgcaacatcattcctctttgagttgtcattcccagcatagtaaactatatgattgtccaattcaaaatggccaatatcagtccatttcagctcactaatgcctaggatatcaatgtttatgcgttccatttcatttttgatgatttctaattttcttagattcatacttcatacattccaggttccgattattaatggatgtttggtaaaggccttgattctttaccaagtgcattcatacccagatttccatctgcctctcctgaagatcctgagatgtcttcagtgcacagagttgggtccccatgccataagtggggaaacaggcccaagagcttgccctgagggttggtggcagggctacaacaccaattcctccctccttgctcatctcatctctgccttctgctcctcggtacatggcccagggtgagctgtttcccacctacgtggacaagctttgtggccgtgacttgatgaacttcccacccttccatgccaatagcatggagaaggacaagctggtggagctgtaccccatcatcacttactttgatacctccttgggcaacatcatggaggaccagaagaccctcaaccccaaagcccagaatctccataccagctgagctccacggcagacaccctttggggatccctcagcaatgtgctctgccacctgtgcaacaagtactacgcaggccacatggacacatcctacagccctgacacctcagtgaaaacatcttacagaacaagaagctgggctgtcagctcctcgtgaagtacaagcagttcatcactgacctagcccaggtcttctag

SEQ ID NO: 62 - protein, 18 aa, synthetic construct
MASFYRLPGILSLLLVLH

SEQ ID NO: 63 - DNA, 1694 bp, synthetic construct
  source 1..1694: mol_type = other DNA
atggttgagtataagcctagatccaggttccagtcggtgggaatagctggtgtcctgcagggagatataagtaggtgagagaagcaggaatagtgaaatagagggagacagagatggtggccatagagctgtaggtggagacgcagtgcatccagcgtgacctgagcctccagctgccggtcccaccgtcctgatcatggcaactttctgccacccccaggaattctgtcattgctgcttgttctacactgggaacgtggggcggggagcccccttcccaacagtcctgacaggaccacctgcaccacacgtcacccatgttccagcaacctcagattccagatcaggaatcaactgctgcagctcaacagtagtgccagcaccctctttattttctgtgtaagttacccctcccaccccccgggactgaggaaggagggtgtgggcctgaagttggagaaatgggcagaattcctacagatatcagtggagcctgcactgacctgggctggaagggaaagcagcgggagggggtgctgtttacaccacacccagctttccccacgcccactcccagctcggactggaatcgactttcccagggttaggtcgggacagagctctgatgagaatgtactgggcctggcacttcaggtattaagattcctccaccatgtggagggagaggcagatggagatacacaaaggagggaaaaggcatggaggtgttcagagaggtgtctttgagaaagacaatgagcagtgggtgtctgggtgcagagagatacccaggtgggtgaaaggacaagtgtgtgtggtgcacctctgaaggtgaggcacccaggttttgtttctttccccatatgaccatgataattaacaaaacaaaaccagttgttactgcatcagttcagactcattgtgaccccatgtgtgtcagagtagaactgcactccatagggtttgcactggttgattttttttgaaatagattgccaggcctttcttctgaagcatctctgggtggacttgaactcccaacctatcagctagaagctgggtacattaaccatttgaccagccaggggctcccaatactaataataagtaataatatcagtccttacatgtccttacataaccaagtgcgttcacacccagatttccatctgacgatcctgagatggcacggagttgggtcctcaggccataggtggggaaactgaagcccaagagcttgccccagaggagggtggcagggctggaacactgagccttcctccttgttcacctcccttcttccctctgctgctcagtacatggaccagggtgagccattcgctatcaaagtaggcaagctgtgaagccctgatacgatagactttctgcccttccatgctgatggcatggagaaggacaggttggtggaggtgtattgcattatcatgtaccttggcacttccctgggcagcatcacccagaacctccacagcaagctgagctccatggggcatatatttcaggggctcctcagcaatgtgccctgccacctgtgcaatgagcaccatatgggccatgtggacacgtcctacagcccttatacctcgggcaagaacatcttacagaagaagctgggctgtcagctcctggggaagtacaagcaggtcattaccaagctgtcccaggccatctag

SEQ ID NO: 64 - protein, 95 aa, synthetic construct
MEKDRLVEVYCIIMYLGTSLGSITQNLHSKLSSMGHIFQGLLSNVPCHLCNEHHMGHVDTSYSPYTSGKNILQKKLGCQLLGKYKQVITKLSQAI

SEQ ID NO: 65 - DNA, 420 bp, synthetic construct
  source 1..420: mol_type = other DNA
atggcccagggtgggccgttccccagcaaagtggacaagctgtcatgcaggcatttctcgacaaacttcccactcttccagggtgatggcacagagaaagacaagctggcagagctctaccaaatcatcacctgctttgatacctccctgggcaatgccatggagtaccaggagaccctcaaccccaaagaccagaacttccacagcaagctgagctccatggaggacatcctgcgggggcttctcagcaacatgctctgctgcctgtgcgactagttccacatgggccacaaggacacatcctacagtccaaatacctcagccaaggatgtcttccggaagaagaagctgggctgtcagctcttggggaagtacaagtatcacggagctggcgctggccttctaggcacaggatattga

SEQ ID NO: 66 - protein, 91 aa, synthetic construct
MAQGGPFPSKVDKLSCRHFSTNFPLFQGDGTEKDKLAELYQIITCFDTSLGNAMEYQETLNPKDQNFHSKLSSMEDILRGLLSNMLCCLCD

SEQ ID NO: 67 - DNA, 618 bp, synthetic construct
  source 1..618: mol_type = other DNA
atggccccattttattggctgcccggcatacttagcttgctgctggttttgcattgggagtatggggccggcaatcccttgcctaacacacccgacaaagctacttgcataactgaccatccctgtgcttccaacctgatgactcaaataaaaaatcaacttgagcagcttaattcatctgccaatacattgttggttctgtattacaccgcacaaggcgagccatttcacaaggaagaggggaaattgtgcggggggcaaaacctcacaaacttcccaccttttcatacaaatgggactgagaaagacaaactggtagaaatctaccagatcatagcctattttaacacctcccttggttcaatcattcaagatcaaaaaacccttaaccctgaggacagaaacttgcactcacagttggattccatagtggacaccctggagggcctcctcagcaatgtattttgtcatttgtgtaatgagtatcaggtgggccaagttgatgtgtactattccccttacacttctgctaaggacatattcaaaaagaagaaactcgggtgtcagttgctggggaagtatgaacaggtgatagccgaattggcccaggatttttga

SEQ ID NO: 68 - protein, 205 aa, synthetic construct
MAPFYWLPGILSLLLVLHWEYGAGNPLPNTPDKATCITDHPCASNLMTQIKNQLEQLNSSANTLLVLYYTAQGEPFHKEEGKLCGGQNLTNFPPFHTNGTEKDKLVEIYQIIAYFNTSLGSIIQDQKTLNPEDRNLHSQLDSIVDTLEGLLSNVFCHLCNEYQVGQVDVYYSPYTSAKDIFKKKKLGCQLLGKYEQVIAELAQDF

SEQ ID NO: 69 - DNA, 618 bp, synthetic construct
  source 1..618: mol_type = other DNA
atggagatcagcgttgaacgaactcacctgcctggcgtcttgctcctgttggtattgcaccaagaacatggtgctagatcactggataccgtcacacccagtaaggttacttacactacctgtcatacatgtcctggaaaccttactacccagatcagaaataaactcaagcagttgaatgggagtgctgacggtttgcttacattgtattgcattgcacaaggggaaccttttcccagcgaggtggacaagatgtgcggctctggtttgaccgactttcccccctttaacgctcacgggcgcaaagaggccatgatgaaggaactctattgcattgtaacatatttgacaacctcccttggaaacattaccagggatcaaaagagcttgaatccccaagatcggaaccttcacggacaattggatttcgtggccactattatgcaacaattgcagtccaacgtgctctgtctgctttacaaggataaggttgaccatgtaggtgagtgctatggccccaacacacttggcaaaacttttttccaaaaaaagagactgggatgtcaagtgttgtttaagtacaaacaagtcattactgagctttatcaagcattctga

SEQ ID NO: 70 - protein, 205 aa, synthetic construct
MEISVERTHLPGVLLLLVLHQEHGARSLDTVTPSKVTYTTCHTCPGNLTTQIRNKLKQLNGSADGLLTLYCIAQGEPFPSEVDKMCGSGLTDFPPFNAHGRKEAMMKELYCIVTYLTTSLGNITRDQKSLNPQDRNLHGQLDFVATIMQQLQSNVLCLLYKDKVDHVGECYGPNTLGKTFFQKKRLGCQVLFKYKQVITELYQAF

SEQ ID NO: 71 - DNA, 615 bp, synthetic construct
  source 1..615: mol_type = other DNA
atgaccagcttcggcagacttcttggaatcctgagtttgctccttattctgcactgggaatatggcactggctccccactctgcaacacacccgataacgccccctgtgtgctcgaccacccctgtgcatctaacataatgacccaaatcagaaatcaactcgctcagctcaacagatccgcaaaggatctttttattgcattcagcaaggcccaaggggaacccttccctagcgatatgcggaagatatgctcccatgggtttattaaattccctcctttccatgcaaacggtacagaaaaagacaagctggtggagctgtatagcattaccgtgttcttcgttttcagtctcgatgacataaccaaattccagaagacatttattcggaaagaccaaaacttgcacagcaaactgaaatctactatggacatacttgccggtctcagaagcaatatgctctgtcgattgtgtagcggtcataatatgcggcctactgatcccatatccggactggacataagtcaaaaggattggttccataaaaagaagctcgggtgcgagttgttgataaagtacgccaggcttattcctgtgcttgctcgggatttctga

SEQ ID NO: 72 - protein, 204 aa, synthetic construct
MTSFGRLLGILSLLLILHWEYGTGSPLCNTPDNAPCVLDHPCASNIMTQIRNQLAQLNRSAKDLFIAFSKAQGEPFPSDMRKICSHGFIKFPPFHANGTEKDKLVELYSITVFFVFSLDDITKFQKTFIRKDQNLHSKLKSTMDILAGLRSNMLCRLCSGHNMRPTDPISGLDISQKDWFHKKKLGCELLIKYARLIPVLARDF

SEQ ID NO: 73 - DNA, 473 bp, synthetic construct
  source 1..473: mol_type = other DNA
atgacccagattaggtcccagatgggacagctcaactcatctgcaaacgatgttttcatcgcttacatgggtcaggggcaacccttcagttccgatgtagatcatatgtgtggtcacggtttgatgtcttttcccccatttcatgctaacggaatcaagcgggagaagttggtcgagttgtatcgcataatggtatattttgactttagtctcgatgatacactcaaatttcaaaagacactttatccaaacgaccaaaatctccattctaagctcaaatctaccatggatatattgccaggattgctgtcaaatgtcttgtgtaggctctgctcaaagaataatatgcgattgaccgacaccatctacggactcaacataagccaggagaacgtgttccataaaaagaaacttgggtgtgagcttctgctcaagtacacccagttgattccttacttggcttgggacttctg

SEQ ID NO: 74 - protein, 157 aa, synthetic construct
MTQIRSQMGQLNSSANDVFIAYMGQGQPFSSDVDHMCGHGLMSFPPFHANGIKREKLVELYRIMVYFDFSLDDTLKFQKTLYPNDQNLHSKLKSTMDILPGLLSNVLCRLCSKNNMRLTDTIYGLNISQENVFHKKKLGCELLLKYTQLIPYLAWDF

SEQ ID NO: 75 - protein, 140 aa, synthetic construct
MTMEDSNGNPLGHNSFKVHICTCPGRHRCTEEDNFRKKWEPCPEPPSGRITKQTLPTSTSSSTKPKKKPLDEKYFTLQIHGHECFKMFLKLNEALELKDAQAGKQPEGSRAQSSLPKSKKRQSTSRHKKLMFKREQPDSD

SEQ ID NO: 76 - protein, 104 aa, synthetic construct
MCNSSCMGSMNPSSPSSHWKTPMVIRWDTTVSRCIFVPVLGDTDVQRKTISTRSGSLALSHPLGGSLSKLCPPAPAPLPSQRRSHWMKNTSPFRSMGMNASRCS

SEQ ID NO: 77 - protein, 54 aa, synthetic construct
MFLKLNEALELKDAQAGKQPEGSRAQSSLPKSKKRQSTSCHKKLMFKREQPDSD

SEQ ID NO: 78 - protein, 48 aa, synthetic construct
MQHCPHLECYSDYSDGLAAPQHLIQVGGILRADICRTPLLFDIVWVTL

SEQ ID NO: 79 - protein, 54 aa, synthetic construct
MFLELNEALELKDAQAGKQPEGSRAQCSLPNSKKGESTTHCKKLMFKREGPDSD

SEQ ID NO: 80 - protein, 128 aa, synthetic construct
MLSSSVPSQKTYPAPIVSVWASCILGQPSPSPTRTPLNLTCCFASWQRPVQCSRGSPQHPRLAPVFTPWPSIRRQHMMEVVKHCRQLECHSDYSDCLDPPQHLMQWEETCMLSIWRTPSLYDTVWGAL

SEQ ID NO: 81 - protein, 120 aa, synthetic construct
MSHQRSVLTTTIHFNFMCNSSCMGSMNPSSPSSHWKTPMVIRWDTTLLRCIFVPVPGDTDVQRKTISTRSGSLALSHPLGGSLSKHCPPAPAPLPSQRRSHWMKNTSPFRSMGMNASRCS

SEQ ID NO: 82 - protein, 54 aa, synthetic construct
MFLKLNEALELKDAQAGKQPEGSRAQSSLPKSKKRQSTSRHKKLTFKREQPDSD

SEQ ID NO: 83 - protein, 52 aa, synthetic construct
MTEVVQHCPHLECCSDYSDGLAAPQHLIQVGGILRADICRTPSLFDIVWGTL

SEQ ID NO: 84 - protein, 116 aa, synthetic construct
MSYLRSVLTTTIHFNFMCSSSSMAGGEPILTIITLEDSDGNLLGHNSFEVHICTVLGETDVQRKKISTTSGSHPLRGSLSKHCTPAPAPLPSQRRSHWMKNISPLRSVGMNASRCS

SEQ ID NO: 85 - protein, 54 aa, synthetic construct
MFLELNEALELKDAQARKQPEGSRAQCSLPNSKKGESTTHCKKLMFKREGPDSD

SEQ ID NO: 86 - protein, 52 aa, synthetic construct
MTEVVQHCPHLECCSDYSDGLAAPQHLIQVGGILHADICRTPSLFDIVWGTL

SEQ ID NO: 87 - protein, 40 aa, synthetic construct
MSYLRSVLTTTIHFNFMCSSSCMGGGGTHPHHHHTGRLRW

SEQ ID NO: 88 - protein, 54 aa, synthetic construct
MFLELNEALELKDAQAGNQPESSRDQCSLPNSKKGESTTHCKKLTFKREGPDSD

SEQ ID NO: 89 - protein, 52 aa, synthetic construct; identical to SEQ ID NO: 83

SEQ ID NO: 90 - protein, 42 aa, synthetic construct
MSYLRSVLTTTIHFNFMCSSSCVGAGNPSSPSSHWKTPMVIC

SEQ ID NO: 91 - protein, 54 aa, synthetic construct
MFLELNEALELKDAQAGKQPEGSRAQCSLPNSKKRESTTHCKKLMFKREGPDSD

SEQ ID NO: 92 - protein, 52 aa, synthetic construct; identical to SEQ ID NO: 83

SEQ ID NO: 93 - protein, 42 aa, synthetic construct; identical to SEQ ID NO: 90

SEQ ID NO: 94 - protein, 54 aa, synthetic construct; identical to SEQ ID NO: 91

SEQ ID NO: 95 - protein, 52 aa, synthetic construct; identical to SEQ ID NO: 83

SEQ ID NO: 96 - protein, 42 aa, synthetic construct
MSYLRSVLTTTIHFNFMCSSSCMAGGNPSSPSSHWKTPMVIC

SEQ ID NO: 97 - protein, 54 aa, synthetic construct
MFLELNEALELKDAQAGKQPEGSRAQCSLPNSKKGESTTHCKKRMFKREGPDSD

SEQ ID NO: 98 - protein, 52 aa, synthetic construct; identical to SEQ ID NO: 83

SEQ ID NO: 99 - protein, 42 aa, synthetic construct; identical to SEQ ID NO: 90

SEQ ID NO: 100 - protein, 54 aa, synthetic construct; identical to SEQ ID NO: 91

SEQ ID NO: 101 - protein, 52 aa, synthetic construct; identical to SEQ ID NO: 83

SEQ ID NO: 102 - protein, 42 aa, synthetic construct
MSYLRSVLTTTIHFNFMCSSSCVGAGNPSSPSSYWKTPMVIC

SEQ ID NO: 103 - protein, 54 aa, synthetic construct; identical to SEQ ID NO: 79

SEQ ID NO: 104 - protein, 128 aa, synthetic construct
MLSSSVPSQKTYPAPIVSVWASCILGQPSPSPTRTPLNLTCCFASWQRPVQCSRGSPQHPRLAPVFTPWPSIRRQHMMEVVKHCRHLECHSDYSDCLDPPQHLMQWEETCMLSIWRTPSLYDTVWGAL

SEQ ID NO: 105 - protein, 120 aa, synthetic construct; identical to SEQ ID NO: 81

SEQ ID NO: 106 - protein, 54 aa, synthetic construct
MFLKLNEALELKDAQAGKQPEGSRAQSSLPKSKKRQSTSRHKQLMFKREQPDSD

SEQ ID NO: 107 - protein, 128 aa, synthetic construct
MLSSSVPSQKTYPATIVSVWASCILGQPSPSPTRTPLNLTCCFASWQRPVQCSRGSPQHPRLAPVFTPWPSIRRQHMMEVVKHCRHLECHSDYSDCLDPPQHLMQWEETCMLSIWRTPSLYDTVWGAL

SEQ ID NO: 108 - protein, 120 aa, synthetic construct; identical to SEQ ID NO: 81

SEQ ID NO: 109 - protein, 54 aa, synthetic construct; identical to SEQ ID NO: 82

SEQ ID NO: 110 - protein, 52 aa, synthetic construct
MTEVMQHCPHLECYSDYSDGLAAPQHLIQVGGILRADICRTPLLFDIVWVTL

SEQ ID NO: 111 - protein, 54 aa, synthetic construct; identical to SEQ ID NO: 79

SEQ ID NO: 112 - protein, 140 aa, synthetic construct
MTMEDSNGNPLGHNSFKVHICTCPGRHRCTEEDNFHKKWEPCPEPPSGRITKQTLPTSTSSSTKPKKKPLDEKYFTLQIHGHECFKMFLKLNEALELKDAQAGKQPEGSRAQSSLPKSKKRQSTSRHKKLMFKREQPDSD

SEQ ID NO: 113 - protein, 140 aa, synthetic construct; identical to SEQ ID NO: 75

SEQ ID NO: 114 - protein, 52 aa, synthetic construct; identical to SEQ ID NO: 110

SEQ ID NO: 115 - protein, 54 aa, synthetic construct; identical to SEQ ID NO: 79

SEQ ID NO: 116 - protein, 104 aa, synthetic construct
MCNSSCMGGRNPSSPSSHWKTPMVIRWDTTVLRCIFVLVLGDTDIQRKTISIRSGSLALSQAWGGSLSEHCPPAPAPLPSQRRSHWMKNTSLFRSMAMNASRCS

SEQ ID NO: 117 - protein, 54 aa, synthetic construct
MFLKLNEALELKDAQAGKQPEGNRAQSSLPKSKKRQSTSRHKKLMFKREQPDSD

SEQ ID NO: 118 - protein, 52 aa, synthetic construct; identical to SEQ ID NO: 110

SEQ ID NO: 119 - protein, 54 aa, synthetic construct; identical to SEQ ID NO: 79

SEQ ID NO: 120 - protein, 52 aa, synthetic construct
MTEVVQHCPHLECCSDYSDGLAAPQHLIQVGGILRANICRTPSLFDIVWGTL

SEQ ID NO: 121 - protein, 42 aa, synthetic construct; identical to SEQ ID NO: 90

SEQ ID NO: 122 - protein, 46 aa, synthetic construct
MRLEDAQAGKQPEGSRAQCSLPNSKKGESTTHCKKLTFKREGPDSD

SEQ ID NO: 123 - protein, 104 aa, synthetic construct
MCNSSCMGGRKPSSPSSHWKTPMVIRWDTTVSRCIFVPALGDADVQRKTISTTSGSLALSHPLGGSLSKHCPPAPAPLPSQRRSHWMKNTSPFRSMGMNVSRCS

SEQ ID NO: 124 - protein, 54 aa, synthetic construct
MFLKLNEALELKDAQAGKQPQGSRAQCSLPKSKKRQSISRHKKIMFKREQPDSD

SEQ ID NO: 125 - protein, 140 aa, synthetic construct
MTMEDSNGNPLGHNSFEVHICTCPGRHRCTEEDNFHNKWEPCPEPPSGRITTQTLPTSTSSSTKPKKKPLDEKYFTLQIHGHECFKMFLKLNEALELKDAQAGKQPEGSRAQSSLPKSKKRQSTSHHKKLMFKREQPDSD

SEQ ID NO: 126 - protein, 140 aa, synthetic construct
MTMEDSNGNPLGHNSFEVHICTCPGKHRCTEEDNFHNKWEPCPEPPSGRITKQTLPTSTSSSTKPKKKPLDEKYFTLQIHGHECFKMFLKLNEALELKDAQAGKQPEGSRAQSSLPKSKKRQSTSRHKKLMFKREQPDSD

SEQ ID NO: 127 - protein, 45 aa, synthetic construct
MTAVVQHCPHLGRCSDYSDGLVPPQHLIQRGENLRAEYLEDTSTL

SEQ ID NO: 128 - protein, 52 aa, synthetic construct
MKWEPCPELPSGWITKRMLPTSTSSSTKPKKPPLDERYFTLQIRGHERYKMF

SEQ ID NO: 129 - protein, 42 aa, synthetic construct
MPRLGSSQRGAGLNSAFPSLRKGNLPPTIKKQTFKREGPDSD

SEQ ID NO: 130 - protein, 19 aa, synthetic construct
MAIYQTSAYDRGHAALPPP

SEQ ID NO: 131 - protein, 52 aa, synthetic construct
MTEVMQHCPHLECCSDYSDGLAAPQHLIQVGEILCADICRTPSLFDIVWGTL

SEQ ID NO: 132 - protein, 42 aa, synthetic construct
MSYLRSALTTTIHFNFMCSSSCKGGRNPSSPSSHWKTPVVIC

SEQ ID NO: 133 - protein, 31 aa, synthetic construct
MPRLGSSQRGAGLNAAFQTLRKGNLPPTVKN

SEQ ID NO: 134 - protein, 135 aa, synthetic construct
MGQGQPFSSDVDHMCGHGLMSFPPFHANGIKREKLVELYRIMVYFDFSLDDTLKFQKTLYPNDQNLHSKLKSTMDILPGLLSNVLCRLCSKNNMRLTDTIYGLNISQENVFHKKKLGCELLLKYTQLIPYLAWDF

SEQ ID NO: 135 - protein, 125 aa, synthetic construct
MRKICSHGFIKFPPFHANGTEKDKLVELYSITVFFVFSLDDITKFQKTFIRKDQNLHSKLKSTMDILAGLRSNMLCRLCSGHNMRPTDPISGLDISQKDWFHKKKLGCELLIKYARLIPVLARDF

SEQ ID NO: 136 - protein, 121 aa, synthetic construct
MCGSGLTDFPPFNAHGRKEAMMKELYCIVTYLTTSLGNITRDQKSLNPQDRNLHGQLDFVATIMQQLQSNVLCLLYKDKVDHVGECYGPNTLGKTFFQKKRLGCQVLFKYKQVITELYQAF

SEQ ID NO: 137 - protein, 85 aa, synthetic construct
MNFPPFHVNGTEREKLVELYHIMDKIVKFQKALHPKDKNLHRKLSSIADTLRGLPNKILCHLCKEYHVSLVDMTYSLSTSERDVS

SEQ ID NO: 138 - protein, 69 aa, synthetic construct
MAQGELFPTYVDKLCGRDLMNFPPFHANSMEKDKLVELYPIITYFDTSLGNIMEDQKTLNPKAQNLHTS

SEQ ID NO: 139 - protein, 25 aa, synthetic construct
MDTSYSPDTSVKTSYRTRSWAVSSS

SEQ ID NO: 140 - protein, 45 aa, synthetic construct
MGHKDTSYSPNTSAKDVFRKKKLGCQLLGKYKYHGAGAGLLGTGY